(19) United States
(12) Patent Application Publication
(10) Pub. No.: US 2021/0283219 A1
(43) Pub. Date: Sep. 16, 2021

(54) INHIBITION OF ALTERNATIVE COMPLEMENT PATHWAY FOR TREATMENT OF RETINAL ISCHEMIC INJURY AND GLAUCOMA

(71) Applicant: Massachusetts Eye and Ear Infirmary, Boston, MA (US)

(72) Inventor: Kip M. Connor, Lexington, MA (US)

(21) Appl. No.: 16/637,992

(22) PCT Filed: Aug. 10, 2018
(86) PCT No.: PCT/US2018/046381

§ 371 Date: Feb. 10, 2020

Related U.S. Application Data
(60) Provisional application No. 62/714,059, filed on Aug. 2, 2018.
(60) Provisional application No. 62/545,305, filed on Aug. 14, 2017.
(60) Provisional application No. 62/544,471, filed on Aug. 11, 2017.

(51) Int. Cl. A61K 38/17; A61P 27/02

(52) U.S. Cl. CPC A61K 38/1725; A61P 27/02

(57) ABSTRACT

Described herein are methods and compositions for reducing or inhibiting retinal cell death in a subject comprising retinal ischemic injury. The method comprises administering to an ischemic retinal tissue of the subject a composition comprising an agent that inhibits or reduces alternative complement pathway activity to reduce or inhibit complement-mediated retinal, neuronal, or retinal neuronal cell death during disease or injury.

### RELATED APPLICATIONS

This application claims the benefit of priority to U.S. Provisional Application No. 62/544,471, filed Aug. 11, 2017, U.S. Provisional Application No. 62/545,305, filed Aug. 14, 2017, and U.S. Provisional Application No. 62/714,059, filed Aug. 2, 2018, the entire contents of each of which are incorporated herein by reference.

### STATEMENT AS TO FEDERALLY SPONSORED RESEARCH

This invention was made with government support under R01-EY027303 awarded by the National Institutes of Health. The government has certain rights in the invention.

### FIELD OF THE INVENTION

The present invention relates to ocular ischemia.

### BACKGROUND

A major component of innate immunity, the complement system is an intricate innate immune surveillance pathway that is able to discriminate between healthy host tissue, diseased host tissue, apoptotic cells and foreign invaders while modulating the elimination and repair of host tissue accordingly. Consisting of serum and tissue proteins, membrane-bound receptors, and a number of regulatory proteins, the complement system is a hub-like network that is tightly connected to other systems. It comprises three key pathways: the classical, lectin and alternative pathways. Within the ocular microenvironment, the alternative complement pathway exhibits low levels of constitutive activation to ensure the intermittent probing of host self-cells, which express inhibitors of complement for protection from activation.

### SUMMARY OF THE INVENTION

The invention features a method for reducing or inhibiting retinal cell death in a subject comprising retinal ischemic injury. The method is carried out by administering to an ischemic retinal tissue of the subject a composition comprising an agent that inhibits or reduces alternative complement pathway activity. For example, retinal cell death comprises neuronal cell death such as death of retinal ganglion cells, e.g., cells located in the inner nuclear layer (INL) of the retina, which cells are critical for visual acuity.

Subjects to be treated include those identified as comprising retinal ischemia or retinal ischemia reperfusion injury. Subjects/patients are identified using standard methods such as imaging of the back of the eye, e.g., using fundus imaging techniques, fluorescein angiography. Such techniques are useful to identify subjects with ischemic injury due to central vein occlusion and/or retinal blockage. Other subjects to be treated include those with ischemic injury or at risk of developing ischemic injury due to glaucoma; such subjects are identified by measuring intraocular pressure (TOP) and detecting a pathologic increase in IOP.

Preferred therapeutic agents include an agent that inhibits or reduces the activity of C3, Factor B (Fb), Factor C5 or Factor D. For example, the agent inhibits or reduces the activity of at least one selected from C3, Factor B (Fb), properdin (Factor p), factor Ba, factor Bb, factor D, C2, C2a, C3a, C3b, C5, C5a, C5b, C6, C7, C8, C9, and C5b-9 in retinal and/or neuronal tissue, e.g., retinal neuronal tissue such as retinal ganglion cells. In another example, the agent inhibits or reduces the activity, transcription stability, translation, modification, localization, cleavage, or function of a polynucleotide or polypeptide encoding any one of the selected from C3, factor B (Fb), properdin (Factor p), factor Ba, factor Bb, factor D, C2, C2a, C3a, C3b, C5, C5a, C5b, C6, C7, C8, C9, and C5b-9 in retinal and/or neuronal tissue. Agents include an antibody or an antigen-binding fragment thereof, a small molecule, a polynucleotide, or a polypeptide. Exemplary agents include a small molecule such as a serine protease inhibitor, a soluble form of a complement receptor, a humanized monoclonal anti-complement antibody or antibody fragment, a complement component inhibitor, a nucleic acid expression vector encoding anti-complement polypeptides, or an anaphylatoxin receptor antagonist.

Administration to the eye is carried out by intraocular injection, intravitreal administration, topical administration, a subconjunctival administration, intranasal administration, intrathecal administration, systemic administration, or directly into the brain. For example, intraocular injection is used to deliver the therapeutic agent to the INL, e.g., to retinal ganglion cells that reside in that layer of the retina. The pathological retinal neuronal cell death is associated with retinal Ischemia reperfusion (IR) injury or glaucoma. For example, ischemia-associated cell death comprises apoptosis, e.g., caspase-mediated apoptosis.

Also within the invention is a composition for reducing or inhibiting retinal cell death in a subject comprising retinal ischemic injury, wherein the composition comprises an agent that inhibits or reduces alternative complement pathway activity. Exemplary formulations are in a water-based solution, e.g., physiologic saline solution. The agent inhibits or reduces the activity of C3, Fb, Factor D, or C5 in neuronal tissue, e.g., the agent inhibits or reduces the activity of at least one selected from C3, Fb, properdin (Factor p), factor Ba, factor Bb, factor D, C2, C2a, C3a, C3b, C5, C5a, C5b, C6, C7, C8, C9, and C5b-9 in retinal, neuronal tissue, or retinal neuronal tissue.

The composition includes an agent that inhibits or reduces the transcription stability, translation, modification, localization, cleavage, or function of a polynucleotide or polypeptide encoding any one of the selected from C3, Fb, properdin (Factor p), factor Ba, factor Bb, factor D, C2, C2a, C3a, C3b, C5, C5a, C5b, C6, C7, C8, C9, and C5b-9 in the target tissue. As described above, the agent comprises an antibody or an antigen-binding fragment thereof, a small molecule, a polynucleotide, or a polypeptide and may include an agent such as a small molecule serine protease inhibitor, a soluble form of a complement receptor, a humanized monoclonal anti-complement antibody or antibody fragment, a complement component inhibitor, a nucleic acid expression vector encoding anti-complement polypeptides, or an anaphylatoxin receptor antagonist. The composition is suitable for intraocular injection, intravitreal injection, intranasal, intrathecal, systemic administration, or directly into the brain and is administered to the subject by a intraocular injection, intravitreal administration, topical administration, a subconjunctival administration. The composition is an ophthalmic composition for ocular administration, e.g, an aqueous formulation as described above which includes therapeutically effective amount of the complement inhibitor agent. Clinical benefit of the treatment determined by an improvement in visual acuity, a decrease in retinal cell death, e.g, a decrease in death of retinal gangion cells, and/or an increase in thickness of the INL. Such parameters are evaluated using standard techniques such as optical coherence tomography to determine the thickness of the INL as well as long term evaluation of changes in visual acuity using standard vision tests. Benefit is also determined by measuring TOP with a decrease in TOP indicating a favorable result of therapy.

Retinal cell death is associated with retinal ischemia reperfusion (IR) injury, optic nerve injury, or glaucoma. As described above, the retinal cell death comprises apoptosis, e.g., caspase-mediated apoptosis of neuronal cells such as retinal ganglion cells in the INL

A pharmaceutical composition for the methods described above is also within the invention. Such a pharmaceutical composition includes a therapeutic agent, e.g., an inhibitor of the alternate complement pathway and a pharmaceutically acceptable carrier. A pharmaceutical acceptable carrier is an inactive ingredient.

Administration of the therapeutic agent is carried out during prior to an ischemic ocular event, at the time of reperfusion of ischemic retinal tissue, within 3 hours of perfusion, within 12 hours of perfusion. For example, subjects with glaucoma are at risk of developing an ischemic ocular event, and in such patients, the alternate complement pathway inhibitor may be administered to the subject prior to an acute ischemic ocular event. For example, the complement inhibitor is administered as an additional therapy with a drug or therapeutic agent to lower TOP.

In some cases, the agent is administered at the time of reperfusion. For example, if the ischemic ocular event is due to a vascular blockage due to a blood clot, the therapeutic agent may be administered in conjunction with administration of an anti-thrombotic drug/agent and or in conjunction with laser therapy administered to break up the blood clot.

The methods and compositions of the invention represent a significant improvement in therapy for subjects. Due to the rapid onset of cell death following an ischemic injury, early intervention is critical to maintaining neuronal survival and thus saving vision. The methods and compositions are therefore administered at the time of diagnosis or as soon as possible thereafter. Immediate or early treatment soon after an ischemic ocular event with alternate complement pathway inhibitors offers significant advantages to prior approaches to treatment and minimizes loss of vision in subject patients.

In other examples, the invention provides compositions and methods for preventing and reducing neuronal cell death associated with neurodegenerative diseases. The methods target the immune system and are broadly applicable to neuronal cell death in both the retina and brain. Retinal ischemia is a major cause of damage in ocular diseases including glaucoma, central retinal vein occlusion and diabetic retinopathy. Ablation and/or inhibition of alternative complement pathway components protects neuronal cells from cell death or reduces the level of neuronal cell death.

Accordingly, compositions for preserving neuronal cells and/or inhibiting or reducing neuronal cell death in a subject comprise an agent that inhibits or reduces complement pathway activity. In particular, the compositions preserve retinal ganglion cells. The complement pathway is the alternative complement pathway or the lectin complement pathway. The agent comprises a small molecule, a polynucleotide, a polypeptide, an antibody or an antibody fragment with means to inhibit or reduce the transcription, transcript stability, modification, localization, secretion, or function of a polynucleotide or polypeptide encoding a component of the alternative or lectin complement pathway. For example, the agent comprises a serine protease inhibitor, a soluble form of a complement receptor, a humanized monoclonal antibody or antibody fragment, a complement component inhibitor, a nucleic acid expression vector encoding an anti-complement agent, a modified complement receptor or an anaphylatoxin receptor antagonist.

Preferably, the agent inhibits or reduces the activity of at least one component of the complement pathway, e.g., the agent inhibits binding of one component to another component of the pathway. Preferably, the complement pathway is the alternative complement pathway. Alternatively, the complement pathway is the lectin complement pathway. The agent inhibits or reduces the activity of at least one component of the alternative or lectin pathway complement pathway. Components of the alternative complement pathway include factor B (Fb), C3, properdin (Factor p), factor Ba, factor Bb, factor D, complement component 2 (C2), complement component 2a (C2a), complement component 3 (C3), complement component 3a (C3a), complement component 5 (C5), complement component 5a (C5a), complement component 6 (C6), complement component 7 (C7), C8, C9, and C5b-9. For example, the inhibitory agent is specific for or binds to a component of the alternate complement pathway as described above. Components of the lectin complement pathway include Mannan-binding lectin serine protease 1 (MASP-1), Mannan-binding lectin serine protease 2 (MASP-2) (also known as Map19), Mannan-binding lectin serine protease 3 (MASP-3), Mannose-binding lectin-associated protein of 44 kDa (Map44), complement component 4 (C4), complement component 4a (C4a), complement component 4b (C4b), C2, C2a and C2b. In a preferred embodiment, the agent specifically binds to the complement pathway component to modulate the transcription, transcript stability, modification, localization, secretion, or function of the component.

For example, the agent that inhibits or reduces the activity of at least one component of the complement pathway is an antibody or an antibody fragment. The antibody or antibody fragment specifically binds to an alternative complement component, such as factor B, C3, properdin (Factor p), factor Ba, factor Bb, factor D, C2, C2a, C3, C3a, C3b C5, C5a, C5b, C6, C7, C8, C9, or C5b-9. The antibody or antibody fragment specifically binds to a lectin complement component, such as MASP-1, MASP-2, MASP-3, Map19, Map44, C4, C4a, C4b, C2, C2a and C2b. The antibody is a monoclonal antibody. The antibody fragment is a antigen-binding (Fab) fragment, a Fab′ fragment, a F(ab′)2 fragment, or an single-chain variable fragment (ScFv) fragment. The antibody is a chimeric antibody. The antibody or antibody fragment is humanized. Preferably, the antibody or fragment thereof is soluble and binds to or inhibits a component or factor of the human alternative complement pathway.

Examples of agents that inhibit or reduce the activity of the complement pathway include, but are not limited to, cinryze, berinert, rhucin, eculizumab, pexelizumab, ofatumumab, TNX-234, compstatin/POT-4, PMX-53, rhMBL, human CD55, BCX-1470, C1-INH, SCR1/TP10, CAB-2/MLN-2222, mirococept, sCR1-sLe^{x}/TP-20, TNX-558, TA106, Neutrazumab, anti-properdin, HuMax-CD38, ARC1905, JPE-1375, and JSM-7717. Examples of agents that inhibit or reduce the activity of the lectin pathway include, but are not limited to, C1-inh, antithrombin, sunflower MASP inhibitors SFMI-1 or SFMI-2, or SMGI inhibitors SGMI-1 or SGMI-2. Some preferred agents that inhibit the complement pathway are disclosed in US Publication 2013/0149373, WO 2013/093762, WO 2010/136311, WO 2009/056631, which are hereby incorporated by reference in their entireties. Other agents are disclosed in Wagner, E. et al., Nature Rev, 2010, 9:43-56, Mucke, H. Et al., IDrugs, 2010, 13(1): 30-37, Ma, K. N. et al., Invest Ophthalmol Vis Sci, 2010, 51(12): 6776-6783, and Ricklin D. et al, Nat Biotech, 2007, 25(11):1265-1275, which are hereby incorporated by reference in their entireties.

The compositions and methods described herein are also useful for inhibiting or reducing neuronal cell death in a subject by inhibiting or reducing complement pathway activity. In particular, the compositions and methods preserve retinal ganglion cells. The compositions inhibit or reduce neuronal cell death by inhibiting or reducing apoptosis. More specifically, the compositions inhibit or reduce caspase-mediated apoptosis. The composition also hinders neovascularization. The subject is a mammal in need of such treatment, e.g., a subject that has been diagnosed with a neuronal cell disorder associated with complement-mediated cell death. For example, the subject has been diagnosed with a neurodegenerative disorder or a predisposition thereto or has been identified as having experienced a head injury such as traumatic brain injury (TBI). For other examples, the subject has been diagnosed with retinal ischemia reperfusion (I/R) injury, retinal detachment, glaucoma or laser-induced corneal neovascularization, and in need of inhibiting or reducing neuronal cell death associated with these indications. The mammal is, e.g., a human, a primate, a mouse, a rat, a dog, a cat, a horse, as well as livestock or animals grown for food consumption, e.g., cattle, sheep, pigs, chickens, and goats. For example, the mammal is a performance mammal, such as a racehorse or racedog (e.g., greyhound). Preferably, the mammal is a human.

The compositions described herein are administered topically, intraocularly, intravitreally, subretinally, or systemically. Intraocular administration is preferred. Also preferred are intravitreal and systemic administration. Preferably, the compositions described herein are administered by intraocular injection or systemically. Accordingly, the composition is formulated as an ophthalmic composition for ocular administration. In a preferred embodiment, the composition is administered shortly after diagnosis of retinal detachment, or appearance of a symptom of retinal detachment. In some aspects, the composition is administered within 1 minute, 5 minutes, 10 minutes, 15 minutes, 30 minutes, 45 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, 30 hours, 36 hours, 42, hours, 48 hours, 56 hours or 72 hours after retinal detachment or appearance of a symptom of retinal detachment. Because of the location of the cell death, the inhibitors are preferably delivered subretinally, e.g., using a fine needle for treatment and reduction of cell death associated with retinal detachment.

The composition comprises a therapeutically effective amount of the agent. In addition, the composition further comprises a pharmaceutically acceptable carrier and/or ophthalmic excipient. Alternatively, a pharmaceutical composition comprises the composition and a pharmaceutically acceptable carrier. Exemplary pharmaceutically acceptable carrier include a compound selected from the group consisting of a physiological acceptable salt, poloxamer analogs with carbopol, carbopol/hydroxypropyl methyl cellulose (HPMC), carbopol-methyl cellulose, carboxymethylcellulose (CMC), hyaluronic acid, cyclodextrin, and petroleum.

Another aspect of the invention provides methods for preserving neuronal cells and/or inhibiting or reducing neuronal cell death in a subject including administering to a neuronal tissue of the subject a composition comprising an agent that inhibits or reduces alternative complement pathway activity, for example, the composition as described above. In particular, the methods preserve retinal ganglion cells. The methods include inhibiting or reducing the transcription, transcript stability, modification, localization, secretion, or function of a polynucleotide or polypeptide encoding a component of the alternative or lectin complement pathway using the agents, such as a small molecule, a polynucleotide, a polypeptide, an antibody or an antibody fragment. Examples of the agent include a serine protease inhibitor, a soluble form of a complement receptor, a humanized monoclonal antibody or antibody fragment, a complement component inhibitor, a nucleic acid expression vector encoding an anti-complement agent, a modified complement receptor or an anaphylatoxin receptor antagonist.

Preferably, the methods include inhibiting or reducing the activity of at least one component of the complement pathway, e.g., inhibiting binding of one component to another component of the pathway. Preferably, the complement pathway is the alternative complement pathway. Alternatively, the complement pathway is the lectin complement pathway. The methods include inhibiting or reducing the activity of at least one component of the alternative or lectin pathway complement pathway. Components of the alternative complement pathway include factor B (Fb), C3, properdin (Factor p), factor Ba, factor Bb, factor D, C2, C2a, C3, C3a, C5, C5a, C6, C7, C8, C9, and C5b-9. For example, the methods include specific inhibition for or inhibitory binding to a component of the alternate complement pathway as described above. Components of the lectin complement pathway include MASP-1, MASP-2, MASP-3, Map19, Map44, C4, C4a, C4b, C2, C2a and C2b. In a preferred embodiment, the methods include specific binding to the complement pathway component to modulate the transcription, transcript stability, modification, localization, secretion, or function of the component.

For example, the methods include the use of an antibody or an antibody fragment that inhibits or reduces the activity of at least one component of the complement pathway. The antibody or antibody fragment specifically binds to an alternative complement component, such as factor B, C3, properdin (Factor p), factor Ba, factor Bb, factor D, C2, C2a, C3, C3a, C3b C5, C5a, C5b, C6, C7, C8, C9, or C5b-9. The antibody or antibody fragment specifically binds to a lectin complement component, such as MASP-1, MASP-2, MASP-3, Map19, Map44, C4, C4a, C4b, C2, C2a and C2b. The antibody is a monoclonal antibody. The antibody fragment is a Fab fragment, a Fab′ fragment, a F(ab′)2 fragment, or an ScFv fragment. The antibody is a chimeric antibody. The antibody or antibody fragment is humanized. Preferably, the antibody or fragment thereof is soluble and binds to or inhibits a component or factor of the human alternative complement pathway. Alternatively, the methods include the use of agents that inhibit or reduce the activity of the complement pathway, as exemplified above.

The methods are also useful for inhibiting or reducing neuronal cell death by inhibiting or reducing complement pathway activity in a subject. In particular, the methods preserve retinal ganglion cells. The methods include inhibiting or reducing apoptosis of neuronal cells. More specifically, the methods include inhibiting or reducing caspase-mediated apoptosis of neuronal cells. The methods also include hindering neovascularization. The methods are, thus, useful to treat a subject that has been diagnosed with a neuronal cell disorder associated with complement-mediated cell death. For example, the subject has been diagnosed with a neurodegenerative disorder or a predisposition thereto or has been identified as having experienced a head injury such as traumatic brain injury (TBI). For other examples, the subject has been diagnosed with retinal ischemia reperfusion (I/R) injury, retinal detachment, glaucoma or laser-induced corneal neovascularization, and in need of inhibiting or reducing neuronal cell death associated with these indications. The subject is, for example, mammal e.g., a human, a primate, a mouse, a rat, a dog, a cat, a horse, as well as livestock or animals grown for food consumption, e.g., cattle, sheep, pigs, chickens, and goats. For example, the mammal is a performance mammal, such as a racehorse or racedog (e.g., greyhound). Preferably, the mammal is a human.

The methods described herein include topical, intraocular, intravitreal, subretinal, or systemic administered. Intraocular administration is preferred. Also preferred are intravitreal and systemic administration. Preferably, the methods described herein include administration by intraocular injection or systemic administration. Accordingly, the methods include ocular administration of an ophthalmic composition. In a preferred embodiment, the methods includes administration shortly after diagnosis of retinal detachment, or appearance of a symptom of retinal detachment. In some aspects, the methods includes administration within 1 minute, 5 minutes, 10 minutes, 15 minutes, 30 minutes, 45 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, 30 hours, 36 hours, 42, hours, 48 hours, 56 hours or 72 hours after retinal detachment or appearance of a symptom of retinal detachment. Because of the location of the cell death, the methods preferably include subretinal delivery of inhibitors, e.g., using a fine needle for treatment and reduction of cell death associated with retinal detachment.

All compounds of the invention are purified and/or isolated. Specifically, as used herein, an “isolated” or “purified” small molecule, nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

An “isolated nucleic acid” is a nucleic acid, the structure of which is not identical to that of any naturally occurring nucleic acid, or to that of any fragment of a naturally occurring genomic nucleic acid spanning more than three separate genes. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybridgene, i.e., a gene encoding a fusion protein. Isolated nucleic acid molecules according to the present invention further include molecules produced synthetically, as well as any nucleic acids that have been altered chemically and/or that have modified backbones. Isolated nucleic acid molecules also include messenger ribonucleic acid (mRNA) molecules.

Although the phrase “nucleic acid molecule” primarily refers to the physical nucleic acid and the phrase “nucleic acid sequence” refers to the linear list of nucleotides of the nucleic acid molecule, the two phrases can be used interchangeably.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to achieve a beneficial clinical effect in a mammal. Ultimately, the attending physician or veterinarian decides the appropriate amount and dosage regimen.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

Figures and slides discussed herein are described below.

FIG. 1A-FIG. 1D are bar graphs showing a significant increase in expression of CD46, CD47, CD55, and CD59.

FIG. 2 is a bar graph showing the results of a cell viability assay.

FIGS. 3A-FIG. 3D are bar graphs showing the results of a quantitative reverse transcription polymerase chain reaction (qRT-PCR) analysis demonstrating a significant decrease in Cd47, Cd55, Cd59a, and Crry.

FIG. 4A-FIG. 4B is a series of images and a bar graph showing the effect of Fb and C3.

FIG. 5A-FIG. 5E are images and a graph showing obstruction of blood flow and the intraocular pressure (TOP) changes during IR surgery.

FIG. 6A-FIG. 6B are images and a bar graph showing IR-induced apoptosis peaks 24 hours after reperfusion.

FIG. 7A-FIG. 7F are bar graphs showing that complement inhibitors gene expression is down-regulated in IR retina.

FIG. 8A-FIG. 8C are images and bar graphs showing that IR-induced retinal cell death and degeneration are suppressed in complement-deficient mice.

FIG. 9A-FIG. 9C are bar graphs showing that complement inhibitors are up-regulated in primary human retinal endothelial cells (HRECs) under shear stress.

FIG. 10A-FIG. 10B are images and bar graphs showing that complement-mediated cell death is suppressed in HRECs by shear stress.

FIG. 11A-FIG. 11B are graphs showing that no gender difference is observed in intraocular pressure (TOP) changes during ischemia reperfusion (IR) surgery.

FIG. 12 is a graph showing that no gender difference is observed in time course of ischemia reperfusion (I/R) induced apoptosis.

FIG. 13 is a graph showing that no gender difference is observed in ischemia reperfusion (I/R) induced apoptosis in complement pathway-deficient mice.

FIG. 14A-FIG. 14C are images and graphs showing the IOP measurement and the IOP trend during the I/R injury model.

FIG. 15 is a graph showing cell death time course in I/R.

FIG. 16A-FIG. 16B are graphs and images showing IOP and retinal ganglion cells (RGCs) in the conventional I/R model of the wild-type (C57BL/6J) mice.

FIG. 17 is a graph showing that membrane bound inhibitors of complement are suppressed in I/R.

FIG. 18A-FIG. 18B are a graph and images showing IOP in the modified ocular hypertension (OH) model of glaucoma.

FIG. 19A-FIG. 19B are a graph and a table showing the disease severity in the modified OH model of glaucoma.

FIG. 20A-FIG. 20C are graphs and images showing the RGC loss in alternative complement deficient mice following OH.

FIG. 21 is a graph showing the RGC loss in alternative complement deficient mice following OH.

FIG. 22 is a graph showing the RGC loss following optic nerve crush (ONC).

FIG. 23A-FIG. 23B are graphs showing the RGC loss in alternative complement deficient mice following ONC.

FIG. 24 is an image showing the retinal architecture.

### DETAILED DESCRIPTION

Several art-recognized models have been used to demonstrate the function of complement mediated cell death of neuronal cells during disease or injury. For example, in addition to an ischemia reperfusion model and retinal detachment model, this mechanism of action is also involved in glaucoma and optic nerve injury. Data from these four models collectively indicate applicability for anti-complement therapeutics in neuronal degeneration.

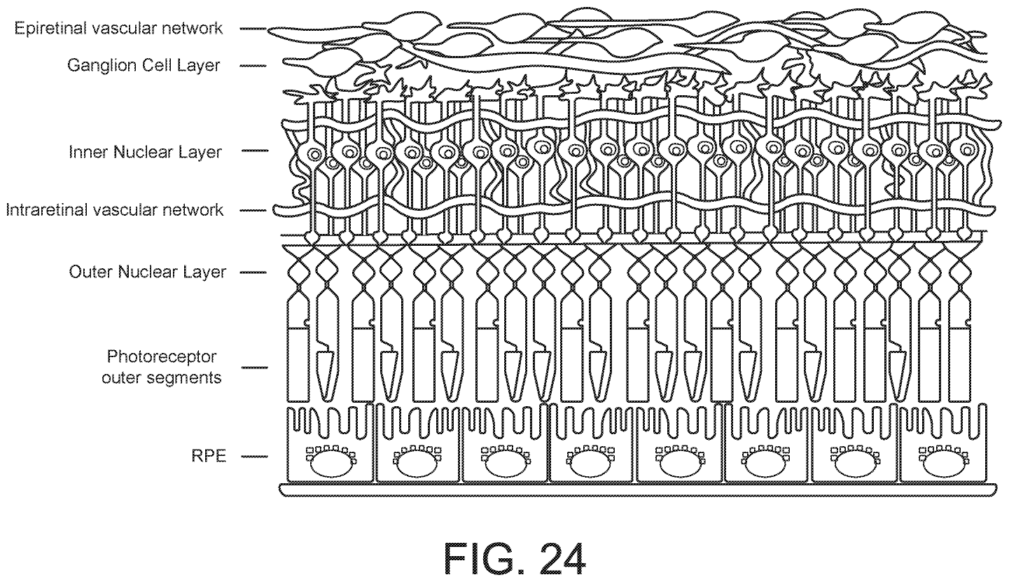

Applicants identified that the alternative complement pathway (e.g., Complement component (C3) and Complement factor B (Fb)) specifically targets retinal ganglion cells for complement mediated removal, for example, in neurodegenerative models. This discovery was unexpected given the unique nature of the disease models assessed and location of retinal ganglion cells within the retinal architecture (see, for example, at FIG. 24). Applicants' previous work discovered that photoreceptor cell death was mediated by the alternative complement (e.g., C3 and Fb) pathway, for example, in a model of retinal detachment, presumably due to exposure of the photoreceptors located in the subretinal layer (outer nuclear layer (ONL)) and deriving its oxygen and nutrients from the choroidal vascular bed to systemic complement because of the break in the blood retinal barrier. See, for example, at U.S. patent application Ser. No. 15/027,917, which is hereby incorporated by reference in their entireties. In this model of retinal detachment, the duration, the extent of injury and height of the retinal detachment played a direct role in cell-targeting by the alternative complement pathway.

By comparison, in neurodegenerative models affecting the retinal ganglion cells, where the blood retinal barrier are intact (models of ischemia reperfusion, glaucoma and optic nerve crush), inhibition of the alternative complement system also facilitates retinal ganglion survival. In these neurodegenerative models, retinal ganglion cell death is initiated by oxidative stress, ischemia and loss of axonal signaling, respectively, each having a unique sequalae that allow for the alternative pathway complement-mediated attack. In contrast to the photoreceptor cell death, the alternative complement pathway-targeting in the retinal ganglion cell death is likely due to production of local complement factors in the retina, as the blood brain barrier is not broken in these models of ocular diseases. Thus, inhibition of this pathway (e.g., C3 and Fb) locally has now broadened our understanding of how this pathway works in the retinal microenvironment under a number of neurodegenerative diseases.

### Complement Regulatory Proteins

The complement system is part of the innate immune system. It is composed of a large number of proteins that interact with one another to directly kill non-self-cells, make them more susceptible to phagocytosis, and recruit inflammatory cells. This pathway includes regulatory proteins, such as complement decay-accelerating factor (also known as DAF or CD55), which disrupts C3 convertase activity, MAC-inhibitory protein (also known as CD59), which prevents C9 from polymerizing and forming the membrane attack complex or membrane attack complex (MAC), and CD46 which is a cofactor of complement factor 1 that cleaves C3b and C4b. This proteins can be membrane bound as well as soluble and free moving. Without these regulatory proteins, complement can become over-activated on self-cells, resulting in their removal or destruction.

Oxygen is a regulator of Cd55 in human umbilical vein endothelial cells. Cd55 is down-regulated in human umbilical vein endothelial cells (HUVECs) under hypoxic conditions and down-regulated on neovessels, which was demonstrated in vivo by Cd55 down-regulated in hypoxic neovessels. This is of importance because there is no blood flow in the neovasculature.

The complement system has been implicated in various diseases, including traumatic brain injury, multiple sclerosis, age-related macular degeneration, diabetic retinopathy, retinal detachment, autoimmune uveitis, Alzheimer, Amyotrophic Lateral Sclerosis (ALS), keratitis, acute lung injury/acute respiratory distress syndrome (ALI/ARDS), ischemia/reperfusion, myocardial infarction, cardiopulmonary bypass, Membranoproliferative glomerulonephritis (MPGN) I & II, systemic lupus erythematosus (SLE), atypical hemolytic uremic syndrome (aHUS), Crohn, ulcerative colitis, celiac disease, rheumatoid arthritis, bone healing, sepsis, MODS (multiple organ dysfunction syndrome), transplantation, infections, C1-inhibitor deficiency, antiphospholipid syndrome, paroxysmal nocturnal hemoglobinuria, gynaecological complications (fetal loss, preeclampsis), etc.

### Neuroimmunity in Ocular Diseases

The complement system plays a role in facilitating cell death in ocular diseases. Examples of the diseases include age-related macular degeneration, diabetic retinopathy, retinal detachment (e.g., the alternative complement pathway targets photoreceptors for cell death) and autoimmune uveitis. Complement-mediated disease are caused by dysregulation of the complement system, which leads to over-activation and the removal or destruction of healthy self-cells. For example, hypoxia in the retina brought on by I/R injury causes complement-mediated neuronal cell death.

Applicants investigated contribution of the complement system in ocular diseases, for example, I/R, Glaucoma, neuvascular diseases, and retinal detachment. The examples of in vivo models and experiments that Applicants employed are described below.

### I/R Models

Blood Supply to the Retina

In the retina, there are two sources of blood flow: the choroidal blood vessels and the central retinal artery. The choroid receives the greatest blood flow (70-80%) and is vital for the maintenance of the outer retinal layers, including the outer plexiform layer (OPL), outer nuclear layers (ONL), photoreceptors, and retinal pigment epithelium (RPE). The remaining 20-30% flows to the retina through the central retinal artery to nourish the inner retinal layers, including the ganglion cell layer (GCL), inner plexiform layer (IPL), and inner nuclear layer (INL).

I/R Injury Pathogenesis

Ischemia is characterized as the disruption of the blood supply to a particular tissue. This prevents the delivery of oxygen and essential nutrients for normal cellular metabolism to the tissue, resulting in cell dysfunction and death. Ischemic injury is worsened once reperfusion occurs. This induces oxidative stress, inflammation, cellular necrosis and apoptosis in the tissue. The pathophysiology of IR injury has been studied extensively, but is still not completely understood.

Retinal I/R Injury/Retinal Ischemia

IR injury in the retina is part of the pathology of many ocular diseases including glaucoma, diabetic retinopathy, and retinal vein occlusion. For example, diabetic retinopathy is observed in approximately 40% of patients with diabetes and a leading cause of vision loss and blindness among working-age adults. Retinopathy of prematurity is observed in premature infants weighing 1250 g or less that are born before 31 weeks and impaired vision or blindness can be developed in severe cases. Acute glaucoma is caused by a sudden increase in pressure inside the eye and immediate treatment is essential to prevent vision loss. Retinal vein occlusion (RVO) is related to compression of the vein due to thickening of the arterial wall and the major prognostic factor for visual outcomes is the extent of initial ischemia.

Currently, the only therapies focus on re-establishing blood flow through the use of anticoagulants, vasodilators, or laser treatment, which are not effective in many cases. The therapies that focus on the injury associated with reperfusion are being investigated, including glutamate receptor and ion channel blockers/inhibitors. However, none of these are clinically available yet. Thus, there still remains a need for a therapy for protecting against damage that arises after reperfusion.

Ischemia Reperfusion (I/R) Injury Models

To investigate complement regulatory protein expression in I/R injury, Applicants used an in vivo murine model of I/R injury. In this model, ischemia is induced by perfusion, followed by reperfusion by re-establishing blood flow. In detail, a 30-gauge needle attached to a line infusing sterile saline is inserted into the anterior chamber. The IOP is then increased by elevating the saline reservoir to 140 cm. Ischemia is examined by assessing the whitening of the anterior segment of the globe and the episcleral veins. After 45 minutes of ischemia, the needle is removed, and reperfusion is examined by observation of the episcleral veins. Applicants took a fundus video during the procedure to evaluate the obstruction of the blood flow.

During the temporary ischemia the intraocular pressure (IOP) is higher than the ocular perfusion pressure, whereas during the natural reperfusion the intraocular pressure (IOP) is lower than the ocular perfusion pressure. IOP is the pressure exerted by the intraocular contents of the eye. The IOP of human is 15.5 mmHg and the IOP of mouse is 12.4 mmHg.

Additionally, Applicants also analyzed the models by using, e.g., RNA extraction, cDNA Synthesis, qRT-PCR, TUNEL staining and quantification and laser capture microdissection (LCM).

### Alternative Complement Pathway Inhibitors

The methods and compositions disclosed herein inhibit or reduce the activity of a complement pathway. Preferably, the complement pathway is the alternative complement pathway. Inhibiting or reducing the activity of a complement pathway, as used herein, refers to the modulation the transcription stability, translation, modification, localization, cleavage, or function of a polynucleotide or polypeptide encoding any one of the selected from C3, Fb, properdin (Factor p), factor B, factor Ba, factor Bb, factor D, C2, C2a, C3a, C5, C5a, C6, C7, C8, C9, and C5b-9. Preferably, the modulation results in an inhibition or decrease in the activity (i.e., function) or expression of the complement component.

Exemplary agents that inhibit or reduce the activity of a complement pathway are described in Table 1 below.

TABLE 1

| | | | Phase of | |
|---|---|---|---|---|
| | | Route of | clinical | |
| DRUG | Mechanism | administration | trial | COMPANY |
| FCFD4514S | Anti-factor D | Intravitreal | 2^{3,4,6} | Genentech |
| | antibody | | | |
| POT-4 | C3 inhibitor | Intravitreal | 2⁵ | Alcon |
| (Compstatin; | | | | |
| APL-1) | | | | |
| APL-2 | C3 inhibitor | Subcutaneous | 2¹⁰ | Apellis |
| | | | | Pharma- |
| | | | | ceuticals, |
| | | | | Inc. |
| AMY 101 | C3 inhibitor | Subcutaneous | 1⁹ | Amyndas |
| | | | | Pharma- |
| | | | | ceuticals |
| ARC1905 | Aptamer-based | Intravitreal | 1^{1,2} | Ophthotec |
| | C5 inh | | completed | |
| Eculizumab | Anti-C5 | oral | 3 | Alexion. |
| (Soliris) | antibody | | | |
| LFG316^{7,8} | Anti-C5 | Intravitreal | | Novartis |
| | antibody | | | |
| TA-106 | Complement | | | Taligen |
| | factor | | | Thera- |
| | B (CFB) | | | peutics, |
| | inhibitor | | | Alexion |
| | | | | Pharma |
| | | | | ceuticals |
| | Anti-factor B | | | Genentech |
| | antibody | | | |

¹Opthotech. ClinicalTrials.gov [Internet]. Bethesda (MD): National Library of Medicine (US). 2000 [Cited 2014 Oct. 7]. Available from http://clinicaltrials.gov/show/NCT00950638 NLM Identifier: NCT00950638.
²Opthotech. ClinicalTrials.gov [Internet]. Bethesda (MD): National Library of Medicine (US). 2000 [Cited 2014 Oct. 7]. Available from http://clinicaltrials.gov/show/NCT00709527 NLM Identifier: NCT00709527.
³Genentech. ClinicalTrials.gov [Internet]. Bethesda (MD): National Library of Medicine (US). 2000 [Cited 2014 Oct. 7]. Available from http://clinicaltrials.gov/show/NCT01229215 NLM Identifier: NCT01229215.
⁴Genentech. ClinicalTrials.gov [Internet]. Bethesda (MD): National Library of Medicine (US). 2000 [Cited 2014 Oct. 7]. Available from http://clinicaltrials.gov/show/NCT00973011 NLM Identifier: NCT00973011
⁵Potentia Pharmaceuticals, Inc. ClinicalTrials.gov [Internet]. Bethesda (MD): National Library of Medicine (US). 2000 [Cited 2014 Oct. 7]. Available from http://clinicaltrials.gov/show/NCT00473928 NLM Identifier: NCT00473928.
⁶Genentech. ClinicalTrials.gov [Internet]. Bethesda (MD): National Library of Medicine (US). 2000 [Cited 2014 Oct. 7]. Available from http://clinicaltrials.gov/show/NCT01602120 NLM Identifier: NCT01602120.
⁷M. Roguska, et al. Generation and Characterization of LFG316, a Fully-Human Anti-C5 Antibody for the Treatment of Age-Related Macular Degeneration [abstract]. In: ARVO 2014 Annual Meeting Abstracts. ARVO Annual Meeting. 2014 May 4-8; Orlando, FL. Abstract No. 3432 - C0281.
⁸A. Carrion, et al. Characterization of the Stoichiometry of Human Complement C5 Binding to LFG316 [abstract]. In: ARVO 2014 Annual Meeting Abstracts. ARVO Annual Meeting. 2014 May 4-8; Orlando, FL. Abstract No. 3432-C0280.
⁹Amyndas Pharmaceuticals. ClinicalTrials.gov [Internet]. Bethesda (MD): National Library of Medicine (US). 2017 [Cited 2018 Aug. 10], Available from https://clinicaltrials.gov/ct2/show/NCT03316521?term=amy+101&rank=1.
¹⁰Apellis Pharmaceuticals, Inc. ClinicalTrials.gov [Internet]. Bethesda (MD): National Library of Medicine (US). 2018 [Cited 2018 Aug. 10]. Available from https://clinicaltrials.gov/ct2/show/NCT03453619?term=APL-2&rank=1.

Several of the therapies described in the above table are antibodies that inhibit or reduce complement pathway activity. Preferably, the antibody specifically binds to the properdin (Factor p), factor B, factor Ba, factor Bb, factor D, C2, C2a, C3a, C3b, C5, C5a, C5b, C6, C7, C8, C9, and C5b-9. In a preferred embodiment, the antibody inhibits the activity (function) of the complement component, for example, preventing or reducing the binding to the cognate receptor, the binding of the receptor to the ligand, cleavage, or activation. Examples of therapeutic antibodies include eculizumab, pexelizumab, ofatumumab, TNX-234, TNX-558, TA106, neutrazumab, and anti-properdin. An exemplary antibody specifically binds to factor B (Fb).

The term “antibody” as used herein includes whole antibodies and any antigen binding fragment (i. e., “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains. CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

“Humanized” forms of non-human (e.g., murine) antibodies are chimeric antibodies which contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992).

Human monoclonal antibodies can be prepared by using trioma technique; the human B-cell hybridoma technique (Kozbor, et al., 1983 Immunol Today 4: 72); and the EBV hybridoma technique to produce human monoclonal antibodies (Cole, et al., 1985 In: Monoclonal Antibodies and Cancer Therapy, Alan R. Liss, Inc., pp. 77-96). Human monoclonal antibodies may be utilized and may be produced by using human hybridomas (Cote, et al., 1983. Proc Natl Acad Sci USA 80: 2026-2030) or by transforming human B-cells with Epstein Barr Virus in vitro (Cole, et al., 1985 In: Monoclonal Antibodies and Cancer Therapy, Alan R. Liss, Inc., pp. 77-96). In addition, human antibodies can also be produced using additional techniques, including phage display libraries. (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); and Lonberg and Huszar, Intern. Rev. Immunol. 13 65-93 (1995).

Human antibodies may additionally be produced using transgenic nonhuman animals which are modified so as to produce fully human antibodies rather than the animal's endogenous antibodies in response to challenge by an antigen (PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

Services are currently offered commercially by companies (i.e., Immunomedics Inc., 300 The American Road, Morris Plains, N.J. 07950, USA; Antitope Ltd., Babraham Research Campus, Babraham, Cambridge CB22 3AT, United Kingdom; and GenScript USA Inc., 860 Centennial Ave., Piscataway, N.J. 08854, USA) for the production of humanized antibodies.

The term “antigen binding portion” of an antibody, as used herein, refers to one or more fragments of an intact antibody that retain the ability to specifically bind to a given antigen (e.g., C3b). Antigen binding functions of an antibody can be performed by fragments of an intact antibody. Examples of binding fragments encompassed within the term “antigen binding portion” of an antibody include a Fab fragment, a monovalent fragment consisting of the VL, VH, CL and CH1 domains; a F(ab)₂ fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al. 1989 Nature 341:544-546), which consists of a VH domain or a VL domain; and an isolated complementarity determining region (CDR).

Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv); see, e.g., Bird et al., 1988 Science 242:423-426; and Huston et al., 1988 Proc. Natl. Acad. Sci. 65:5879-5883). Such single chain antibodies include one or more “antigen binding portions” of an antibody. These antibody fragments are obtained using conventional techniques known to those of skill in the art, and the fragments are screened for utility in the same manner as are intact antibodies. Antigen binding portions can also be incorporated into single domain antibodies, maxibodies, minibodies, interbodies, diabodies, triabodies, totrabodies. v-NAR and bis-scFv (see, e.g., Hollinger and Hudson. 2005. Nature Biotechnology, 23, 9, 1126-1136).

Antigen binding portions can be incorporated into single chain molecules comprising a pair of tandem Fv segments (VH-CH1-VH-CH1) which, together with complementary light chain polypeptides, form a pair of antigen binding regions (Zapata et al., 1995 Protein Eng. 8(10): 1057-1062; and U.S. Pat. No. 5,641,870).

The term “binding specificity” or “specifically binds” as used herein refers to the ability of an individual antibody combining site to react with only one antigenic determinant, and therefore does not bind other complement components. The combining site of the antibody is located in the Fab portion of the molecule and is constructed from the hypervariable regions of the heavy and light chains. Binding affinity of an antibody is the strength of the reaction between a single antigenic determinant and a single combining site on the antibody. It is the sum of the attractive and repulsive forces operating between the antigenic determinant and the combining site of the antibody.

To screen for antibodies which bind to a particular epitope on the antigen of interest, a routine cross-blocking assay such as that described in Antibodies, A Laboratory Manual, Cold Spring Harbor Laboratory, Ed Harlow and David Lane (1988), can be performed. Alternatively, epitope mapping, e.g. as described in Champe et al. (1995) J. Biol. Chem. 270:1388-1394, can be performed to determine whether the antibody binds an epitope of interest, as described in WO2009061910A1 (incorporated in its entirety by reference herein). Example antibodies from Table 1 above are described briefly below.

### Anti-Factor B Antibodies

Anti-factor B antibodies are selected using a factor B antigen derived from a mammalian species. Preferably the antigen is human factor B. However, factor Bs from other species such as murine factor B can also be used as the target antigen. The factor B antigens from various mammalian species may be isolated from natural sources. In other embodiments, the antigen is produced recombinantly or made using other synthetic methods known in the art. The antibody selected will normally have a sufficiently strong binding affinity for the factor B antigen. For example, the antibody may bind human factor B with a Kd value of no more than about 5 nM, preferably no more than about 2 nM, and more preferably no more than about 500 pM.

Antibody affinities may be determined by a surface plasmon resonance based assay (such as the BiAcore assay as described in Examples); enzyme-linked immunoabsorbent assay (ELISA); and competition assays (e.g. RIA's), for example. Also, the antibody may be subject to other biological activity assays, e.g., in order to evaluate its effectiveness as a therapeutic. Such assays are known in the art and depend on the target antigen and intended use for the antibody. Examples include the HUVEC inhibition assay (as described in the Examples below); tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and in vitro and in vivo assays described below for identifying factor B antagonists.

To screen for antibodies which bind to a particular epitope on the antigen of interest, a routine cross-blocking assay such as that described in Antibodies, A Laboratory Manual, Cold Spring Harbor Laboratory, Ed Harlow and David Lane (1988), can be performed. Alternatively, epitope mapping, e.g. as described in Champe et al. (1995) J. Biol. Chem. 270:1388-1394, can be performed to determine whether the antibody binds an epitope of interest. In a preferred embodiment, the anti-factor 13 antibodies are selected using a unique phage display approach. The approach involves generation of synthetic antibody phage libraries based on single framework template, design of sufficient diversities within variable domains, display of polypeptides having the diversified variable domains, selection of candidate antibodies with high affinity to target factor B antigen, and isolation of the selected antibodies.

Examples of anti-factor B antibodies are described in WO2009061910A1, WO2013177035, WO2008140653, and US 20050260198 (each of which is hereby incorporated by reference).

### Anti-Factor Bb

An exemplary antibodies specific for factor B for use in inhibiting the activity of C3bBb or PC3bBb complexes, such an antibody inhibits the proteolytic activity of factor B in C3/C5 convertases. Anti-Factor Fb antibodies selectively block the binding of factor Bb to the PC3bB complex without inhibiting classical pathway activation as described in WO2013152020 (incorporated by reference in its entirety herein).

### Anti-Factor D

Further example therapies comprise antibodies that target factor D. Complement factor-d (Fd) is a serine protease, which cleaves Fb once bound to C3b resulting in the assembly of the alternative pathways C3-convertase. An example anti-Factor D antibody comprises light chain HVR-1 comprising ITSTDIDDDMN (SEQ ID NO: 1), light chain HVR-2 comprising GGNTLRP (SEQ ID NO: 2), and light chain HVR-3 comprising LQSDSLPYT (SEQ ID NO: 3) and may comprise amino acid substitutions provided that they preserve its antibody binding properties, e.g., as described in US 20140065137, incorporated by reference in its entirety herein. An additional exemplary factor D antibody or a binding fragment thereof may bind to the same epitope on human Factor D as monoclonal antibody 166-32 and is produced by the hybridoma cell line deposited under ATCC Accession Number HB-12476 as described in U.S. Pat. No. 8,124,090, incorporated in its entirety by reference herein. Another exemplary anti-factor D antibody is U.S. Pat. No. 8,372,403 (incorporated in its entirety by reference herein). A further example of Factor D neutralizing antibody includes, but is not limited to, is the Fd antibody from R&D systems, MAB5430.

### Anti-C3 Antibody

An example anti C3 antibody selectively blocks the binding of factor B to C3b without inhibiting the classical pathway activation. These types of antibodies, as described in WO2013152024 do not inhibit the interaction of C3b to C5 and therefore have a unique function in inhibiting the alternative pathway. Alternative anti-C3 antibodies can inhibit complement activation by way of inhibition of C3b function, as described in US 20100111946. Each of the references are herein incorporated by reference in their entireties. An example of C3 neutralizing antibody includes, but is not limited to, the C3 antibody from Abcam, ab11862.

### Anti-Properdin Antibody

An exemplary anti-Properdin antibody is that described in WO 2013006449 (incorporated in its entirety by reference herein) with the epitope comprising amino acids of the sequence: RGRTCRGRKFDGHRCAGQQQDIRHCYSIQHCP (SEQ ID NO: 4).

### Anti-C5 Antibody

The prevention of C5a generation with antibodies during the arrival of sepsis in rodents has been shown to greatly improve survival; while related findings were made when the C5a receptor (C5aR) was blocked, using either antibodies or a small molecular inhibitor (Landes, U., et al., Anti-c5a ameliorates coagulation/fibrinolytic protein changes in a rat model of sepsis. American Journal Of Pathology, 2002. 160(5): p. 1867; Riedemann, N. C., R. F. Guo, and P. A. Ward, A key role of C5a/C5aR activation for the development of sepsis. Journal of Leukocyte Biology, 2003, 74(6): p. 966). An additional exemplary anti-C5 antibody is described in WO1995029697. An additional example antibody is the monoclonal antibody designated MAb137-26, which binds to a shared epitope of human C5 and C5a, as described in U.S. Pat. No. 8,372,404. Each of the references are herein incorporated by reference in their entireties.

In some embodiments, the agent comprises a small molecule, a polynucleotide, a polypeptide, with means to inhibit or reduce the transcription, transcript stability, modification, localization, secretion, or function of a polynucleotide or polypeptide encoding a component of the alternative or lectin complement pathway. For example, the agent comprises a serine protease inhibitor, a soluble form of a complement receptor, a complement component inhibitor, a nucleic acid expression vector encoding an anti-complement agent, a modified complement receptor or an anaphylatoxin receptor antagonist.

Preferably, the agent inhibits or reduces the activity of at least one component of the complement pathway, e.g., the agent inhibits binding of one component to another component of the pathway. Preferably, the complement pathway is the alternative complement pathway. The agent inhibits or reduces the activity of at least one component of the alternative or lectin pathway complement pathway. Components of the alternative complement pathway include factor B (Fb), C3, properdin (Factor p), factor Ba, factor Bb, factor D, C2, C2a, C3, C3a, C5, C5a, C6, C7, C8, C9, and C5b-9. For example, the inhibitory agent is specific for or binds to a component of the alternate complement pathway as described above. Components of the lectin complement pathway include MASP-1, MASP-2, MASP-3, Map19, Map44, C4, C4a, C4b, C2, C2a and C2b. In a preferred embodiment, the agent specifically binds to the complement pathway component to modulate the transcription, transcript stability, modification, localization, secretion, or function of the component.

Examples of agents that inhibit or reduce the activity of the complement pathway include, but are not limited to, cinryze, berinert, rhucin, eculizumab, pexelizumab, ofatumumab, TNX-234, compstatin/POT-4, PMX-53, rhMBL, human CD55, BCX-1470, C1-INH, SCR1/TP10, CAB-2/MLN-2222, mirococept, sCR1-sLe^{x}/TP-20, TNX-558, TA106, Neutrazumab, anti-properdin, HuMax-CD38, ARC1905, JPE-1375, and JSM-7717. Examples of agents that inhibit or reduce the activity of the lectin pathway include, but are not limited to, C1-inh, antithrombin, sunflower MASP inhibitors SFMI-1 or SFMI-2, or SMGI inhibitors SGMI-1 or SGMI-2. Some preferred agents that inhibit the complement pathway are disclosed in US Publication 2013/0149373, WO 2013/093762, WO 2010/136311, WO 2009/056631, which are hereby incorporated by reference in their entireties. Other agents are disclosed in Wagner, E. et al., Nature Rev, 2010, 9:43-56, Mucke, H. Et al., IDrugs, 2010, 13(1): 30-37, Ma, K. N. et al., Invest Ophthalmol Vis Sci, 2010, 51(12): 6776-6783, and Ricklin D. et al, Nat Biotech, 2007, 25(11):1265-1275, which are hereby incorporated by reference in their entireties.

Other agents that inhibit or reduce complement pathway activity are serine protease inhibitors. The complement cascade relies upon the consecutive cleavage and activation of several proteases. Proteases in the complement cascade include, for example, C1r, C1s, C2a, MASP1, MASP2, factor D, and factor B. The protease inhibitor binds to the protease and preferably prevents its cleavage function. Examples of serine protease inhibitors are C1-Inh and rhucin.

Soluble complement regulators are also useful as agents that inhibit or reduce complement pathway activity. For example, the agent is a soluble form of a complement receptor that competes with endogenous complement receptors, thereby reducing the complement pathway activity. Alternatively, the agent is a soluble form of an endogenous complement inhibitor that reduces complement pathway activity. For example, the agent is a soluble form of DAF/CD55 of CD59. Examples of soluble complement regulators include sCR1/TP10, CAB-2/MLN-2222, mirococept, and soluble CD55 mimetic.

Another agent that inhibits or reduces complement pathway activity is a complement component inhibitor, such as a small molecule that interrupts protein functions by steric hindrance or induction of conformational changes. The agent may be a peptide, a nucleotide, or a synthetic molecule. For example, the agent may be an aptamer, which is a single-stranded nucleotide that has molecular recognition properties similar to those of antibodies but can be selected in an automated high-throughput process known as SELEX. Aptamers that recognize complement components can provide complete blockage of downstream complement activation. For example, anti-C5 aptamer (ARC1905) features a subnanomolar binding affinity for C5 and inhibits the cleavage of C5a and C5b. Other examples of complement component inhibitors include compstatin/POT-4.

Anaphylatoxin receptor antagonists can also be used to inhibit or reduce complement cascade signaling. Anaphylatoxins C3a and C5a are potent inflammatory mediators that binds to high affinity receptors. These antagonists are designed to bind to the receptor with high affinity without inducing any signaling activity, thereby inhibiting and reducing complement pathway activity. Examples of anaphylatoxin receptor antagonists include PMX-53, PMX-205, JPE-1375, and JSM-7717.

Nucleic acid expression vectors that encodes an anti-complement agent are also useful for inhibiting or reducing complement pathway activity. The anti-complement agent may be a polynucleotide or a polypeptide that inhibits or reduces a complement component activity or expression. The polynucleotide may be an interfering RNA or an aptamer. The polypeptide may be a complement inhibitor or receptor antagonist. For example, the agent is an AAV-expression vector comprising CD55.

Non antibody inhibitors are also listed in Table 1 above, brief descriptions of these inhibitors are found below.

### POT-4

POT-4 is a derivative of the cyclic peptide Compostatin. It is capable of binding to human complement factor C3 (Potentia pharmaceuticals, http://www.potentiapharma.com/products/pot4.htm). POT 4 suppresses complement activation by preventing the formation of key elements within the proteolytic cascade, thus impeding local inflammation, upregulation of angiogenic factors and subsequent tissue damage (O. S. Punjabi and P. K. Kaiser, Review of Ophthalmology. Oct. 4, 2012. http://www.reviewofophthalmology.com/content/d/retinal_insider/c/36952).

Derivatives of the compstatin family (e.g., AMY-101, Amyndas Pharmaceuticals) are currently the only low molecular weight inhibitors of C3 that have advanced through clinical development for the treatment of several indications. AMY-101 is has been granted Orphan Designation by both the EMA and US FDA for the treatment of C3 glomerulopathy (C3G).

APL-2 is PEGylated APL-1 formulated for subcutaneous (PNH) and intravitreal administration (AMD). APL-2 is being trialled alone and as an add-on to the approved PNH therapy eculizumab (Phase 1 trial NCT02264639).

### ARC 1905

ARC 1905 is a PEGylated, stabilized aptamer targeting complement factor C5, blocking the cleavage of C5 into C5a and C5b fragments (ARC1905 inhibits C5—Dry/Wet AMD Intravitreal, http://www.amdbook.org/content/arc1905-inhibits-c5-drywet-amd-intravitreal). Like POT-4, it is similarly selective for a centrally positioned component within the cascade, although exerting its effect further downstream (O. S. Punjabi and P. K. Kaiser, Review of Opthomology. Oct. 4, 2012. http://www.reviewofophthalmology.com/content/d/retinal_insider/c/36952).

### TA-106

TA-106 inhibits Factor B, a serine proteinase that is unique to the alternative pathway and exists upstream of complement proteins targeted by many other drugs, including complement 3 (C3) and C5. (BioCentury, The Bernstein Report on BioBusiness. Aug. 13, 2007). It is primarily being investigated as an inhaled formulation in the treatment of severe, chronic asthma refractory to current therapies and is recently being studied for macular degeneration (O. S. Punjabi and P. K. Kaiser, Review of Ophthalmology. Oct. 4, 2012. http://www.reviewofophthalmology.com/content/d/retinal_insider/c/36952).

Non-limiting examples of non antibody inhibitors of C3 and factor B also include: Peptide Cp40 [PMID: 25982307]

Cp40 is a 14 amino acid analogue of the cyclic peptide compstatin. Like compstatin, Cp40 is an inhibitor of complement component C3. Cp40 has shown effective C3 inhibition in C3 glomerulopathy (C3G) in vitro. C3 glomerulopathies are a group of ultra-rare renal diseases caused by uncontrolled activation of the alternative complement pathway, for which there are no disease-specific treatments (http://www.guidetopharmacology.org/GRAC/LigandDisplayForward?ligandId=9220).

### Complin

Complin is a novel cyclic hendecapeptide that inhibits activation of factor B. Complin is suggested to inhibit factor B activation primarily by inhibiting its cleavage by factor D (Kadam et al. J Immunol Jun. 15, 2010, 184 (12) 7116-7124; DOI: https://doi.org/10.4049/jimmunol.1000200).

### Compound 51 [PMID: 19743866]

Compound 51 ((2S)-2-[[(2S)-2 [[(2S)-2-[[(2S)-[[(2S)-2-[[(2S)-2-acetamido-5-(diaminomethylideneamino)pentanoyl]amino]-4-methylpentanoyl]amino]-4,4-dimethylpentanoyl]amino]-4-methylpentanoyl]amino]propanoyl]amino]-5-(diaminomethylideneamino)pentanoic acid; Ac-RLTbaLAR-H) is a hexapeptide which potently inhibits complement factor B (http://www.guidetopharmacology.org/GRAC/LigandDisplayForward?ligandId=8653).

Non-limiting examples of non antibody inhibitors of C3 and factor B further include C3 specific nanobody (Jensen, R. K. et al., J Biol Chem. 2018; 293(17):6269-6281), Fb-specific small molecules (U.S. Pat. No. 9,475,806), and Fb-specific antisense oligonucleotide (Grossman, T. R. et al. Immunobiology, 2016; 221(6):701-708), which are hereby incorporated by reference in their entireties.

### Example 1: Ischemia Reperfusion Model

Ischemia reperfusion (I/R) injury is associated with retinal diseases such as acute angle glaucoma, retinal vein occlusion, and diabetic retinopathy. I/R injury is caused by oxygen and nutrient deprivation due to a lack of blood flow to the tissues. Injury is compounded when normal blood flow is reestablished due to an increase in oxidative stress, cellular necrosis, apoptosis, and inflammation. Current treatments for I/R injury include anti-VEGF therapeutics and panretinal photocoagulation to prevent neovascularization. There remains a need for therapeutics to reduce retinal damage and cell death, e.g., neuronal cell death, from reperfusion injury. The compositions and methods described herein provide a solution to the problems and drawbacks of earlier methods.

The complement cascade is part of the innate immune system and inflammation response. The complement system is comprised of a variety of different proteins that work together to induce an inflammatory response to remove nonfunctional self-cells and non self-cells. The system is comprised of the classical, lectin, and alternative pathways. It is in a constant state of low-level activation, but over activation can lead to diseases such as age-related macular degeneration, diabetic retinopathy, and uveoretinitis. Full activation of the complement system leads to the formation of the MAC, which causes cell lysis and death. The level of activation is regulated by proteins such as the MAC-inhibitory complex (CD59) and decay-accelerating factor (CD55). CD59 prevents complement protein 9 from polymerizing and completing the formation of the MAC. CD55 disrupts C3 and C5 convertase formation and accelerates their decay.

The role of the complement cascade in I/R injury has been investigated in a variety of tissues including the kidney, intestine, lung, heart, and more. However prior to the invention, the role of complement in the retinal I/R injury has not been as extensively investigated. and needs to be further understood. A significant reduction in retinal ganglion cell loss and overall damage grade was found in C3−/− mice compared to control mice one week following induction of I/R. An increased expression of C3, in C3 sufficient mice, was observed in the retina after three weeks following ischemia compared to control eyes.

The major pathology of I/R is the reduction of oxygen, or hypoxia, caused by the total absence of bloodflow. The cellular transcriptional response to hypoxia has been well documented. The regulation of complement inhibitors by hypoxia indicated that CD55 is down regulated in HUVECs under hypoxic conditions. Cd55 is also down regulated in mouse neovessels, which have an absence of bloodflow. The flow of blood over the endothelial cells lining blood vessels induces a force known as shear stress. The regulation of complement inhibitor CD59 by shear stress has been demonstrated in vitro. These results indicated that CD55 and CD59 is down regulated in response to I/R injury due to the combination of hypoxia and lack of shear stress, thus leading to injury caused by the over activation of complement on healthy self-cells, and subsequently leading to cell death.

The following materials and methods were used to generate the data described herein.

### Cell Culture

Primary human retinal endothelial cells (HRECs) were purchased from Cell Sytems (ACBRI 181) and grown in EGM-2 Growth Medium with SingleQuots (Bulletkit CC-3162; Lonza) supplemented with 1% L-glutamine and penicillin-streptomycin. They were grown to confluence in T75s coated with 0.2% gelatin under the following incubator conditions: 5% CO2, 37° C., and 95% humidity. HRECs used for experiments were P7 to P9.

### In Vitro Shear Stress Model

Primary human retinal endothelial cells (HRECs) were seeded into three wells of a 6-well plate at a density of 500,000 cells/well. For each experiment, one plate was used for each magnitude of shear stress. Cells were grown to confluence on 6-well plates. Once confluent, the cells were incubated in starvation medium (EBM-2 basal medium (CC-3156), 5% calf serum, 25 mM HEPES, L-glutamine, and penicillin-streptomycin) for 24 hours to synchronize their cell cycles. Fresh starvation medium was then added and the plates were placed on a shaker set to 150 rpm (to mimic 5 dynes/cm2), 240 rpm (to mimic 10 dynes/cm2), or the shelf of the incubator as a static control (0 dynes/cm2) for 24 hours. Cells of the same passage were placed in the same incubator as the static control. Shear stress was approximated using the formula τ_max=α√(ρη[(2πf))]\hat{ }3), where α is the radius of orbital rotation (0.95 cm), ρ is the density of the medium (1.0 g/ml), η is the density of the medium (7.5×10-3 dynes·s/cm²), and f is the frequency of rotation (rotations/second).

### Live/Dead Assay

To investigate if shear stress has a protective effect towards complement mediated cell death, HRECs were cultured in starvation medium supplemented with 1% mouse serum, while being exposed to shear stress. The percentage of dead cells was determined using the ReadyProbes Cell Viability Imagining Kit (ThermoFisher Scientific, Waltham, Mass.). Three representative images of the peripheral area were taken per well using Zeiss Zen imaging software. Without any post processing, Fiji image analysis software was used to automatically count the number of dead cells (green) and total cells (blue) using the particle analyzer tool. The number of dead cells and total cells of the three images taken for each well were averaged to calculate the percent cell death per well.

### Animals

Eight-week-old male C57BL/6J mice from Jackson Laboratories (Bar Harbor, Me., USA) were used for all experiments. The Fb−/− mice were also used in the studies described herein.

### Mouse Model of Retinal Ischemia-Reperfusion

C57BL/6J weighing 23-25 g were anesthetized (T4, 840-2; Avertin; 2,2,2 tribromoethanol; Sigma-Aldrich, St. Louis, Mo., USA, in isoamyl alcohol, A730-1; Thermo Scientific, Waltham, Mass., USA). The anterior chamber of the left eye was cannulated with a 30-gauge needle connected to a sterile saline reservoir elevated 140 cm. Ischemia was examined by blanching of the episcleral veins and sustained for 45 min while maintaining an IOP of 80-90 mmHg. IOP was measured 5 min and 20 min after induction of ischemia using a TonoLab tonometer (icare, Vantaa, Finland). Reperfusion occurred immediately upon removal of needle, which was examined by observation of the episcleral veins.

### TUNEL Cell Death Assay

TUNEL cell death assay was carried out using standard methods.

RNA Isolation and qRT-PCR

For the shear stress model, cells were trypsinized, pelleted by centrifugation, and flash frozen in liquid nitrogen before being stored at −80° C. The cells were homogenized and total RNA was collected using the RNeasy Mini Kit (Qiagen) following the manufacturers instructions. Total RNA was measured using the NanoDrop 2000 Spectrophotometer (ThermoFisher Scientific). cDNA was normalized prior to reverse transcription with SuperScript III and random hexamer primers (Invitrogen). Quantitative Real-time PCR was performed using cDNA and a StepOnePlus Real-Time PCR System (Applied Biosystems). CD55 (Hs00892618_m1, ThermoFischer Scientific), CD59 (Hs00174141_m1; ThermoFischer Scientific), and β-actin (4333762F, ThermoFisher Scientific) probes were used in combination with TaqMan Universal PCR Master Mix (4304437; ThermoFisher Scientific). All data was normalized to β-actin.

For whole retina gene expression analysis eyes were enucleated, then whole retina was isolated and flash frozen in liquid nitrogen before being stored at −80° C. The retinas were homogenized using pestles and total RNA was collected using the RNeasy Mini Kit (Qiagen) following the manufacturers instructions. Total RNA was measured using the NanoDrop 2000 Spectrophotometer (ThermoFisher Scientific). cDNA was normalized prior to reverse transcription with SuperScript III and random hexamer primers (Invitrogen). Quantitative Real-time PCR was performed using cDNA and a StepOnePlus Real-Time PCR System (Applied Biosystems). Cd55 (Mm00438377_m1, ThermoFischer Scientific), Cd59a (Mm00483149_m1, ThermoFischer Scientific), Cd46 (Mm00487625_m1, ThermoFischer Scientific), Cd47 (Mm00495011_m1, ThermoFischer Scientific), and Crry probes were used in combination with TaqMan Universal PCR Master Mix (4304437; ThermoFisher Scientific). All data was normalized to β-actin.

### Statistical Analysis

Data was analyzed using one-way ANOVA. Results are expressed as mean±SEM. Significance was set at P<0.05.

### Inhibitors of Complement in I/R Injury

Shear stress is a frictional force that acts on the endothelial cells of vessels in response to blood flow. To determine the impact of shear stress on complement inhibitor expression, a model of sheer stress was used to demonstrate that expression of complement regulatory proteins CD46, CD47, CD55, and CD59 is regulated by shear stress in HRECs. In detail, primary human retinal endothelial cells were cultured to confluence in 6-well plates before being incubated in starvation medium for 24 hours. Medium containing 1% mouse serum was then added to the 6-well plates as a source of foreign complement, and the cells were incubated on a shaker set to 150 rpm (to mimic 5 dynes/cm2), 240 rpm (to mimic 10 dynes/cm2), or the shelf of the incubator as a static control, exposing HRECs to orbital shear stress (0, 5, or 10 dynes/cm2) for 24 h.

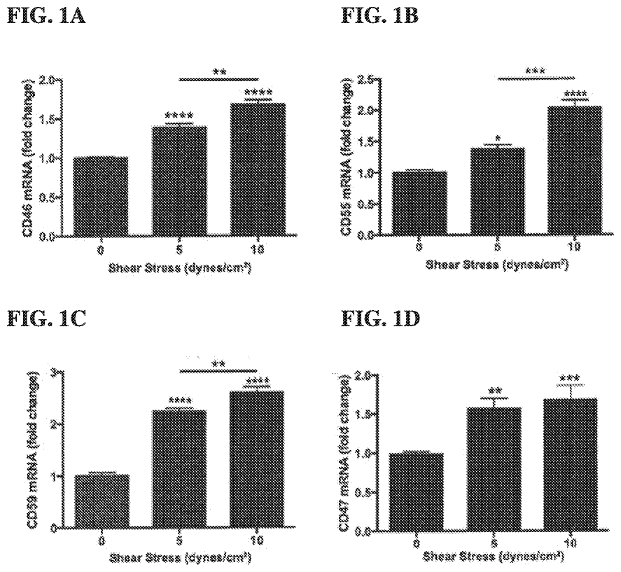

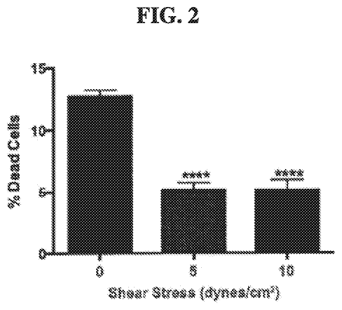

qRT-PCR results (FIGS. 1A-D) showed a significant increase in expression of CD46, CD47, CD55, and CD59. The increase in expression of CD55 and CD59 is proportional to the magnitude of shear stress, with a significant difference (CD55 p=0.002; CD59 p=0.0097) between the fold change of 5 and 10 dynes dynes/cm2. The expression of CD47 is not proportional to the magnitude of shear stress. This result is important because it shows that normal vascular blood flow is important for endothelial cell homeostasis. If blood flow is reduced or stopped then so is shear stress and this will lead to a reduction of complement regulator production. This in turn increases complement activation on endothelial cells leading causing damage or apoptosis.

### Shear Stress is Protective Against Complement Mediated Cell Death:

Studies were carried out to investigate whether the increase in complement inhibitor expression by shear stress would have a protective effect against complement-mediated cell death. HRECs were incubated in media supplemented with 1% mouse serum while being exposed to shear stress. The complement in the mouse serum will recognize the HRECs as non-self cells and target them for destruction by formation of the MAC complex. If the cells exposed to shear stress are synthesizing more complement inhibitor proteins, these cells should survive by inactivating complement factors on their surface, disrupting the formation of the MAC complex. To quantify this, after 24 hours cells were stained with a viability imaging kit that stained all cell nuclei blue and stained cells with compromised plasma membrane integrity, or dead cells, green (e.g., ReadyProbes Cell Viability Imagining Kit). Three images were taken per well in the peripheral area. Cell death images were quantified using ImageJ and the percentage of dead cells was calculated after 24 h. Results showed cell death was significantly increased by 2-fold (FIG. 2) in the static control plate compared to both the shear stress plates. Indicating that shear stress does help to protect HRECs from complement mediated cell death.

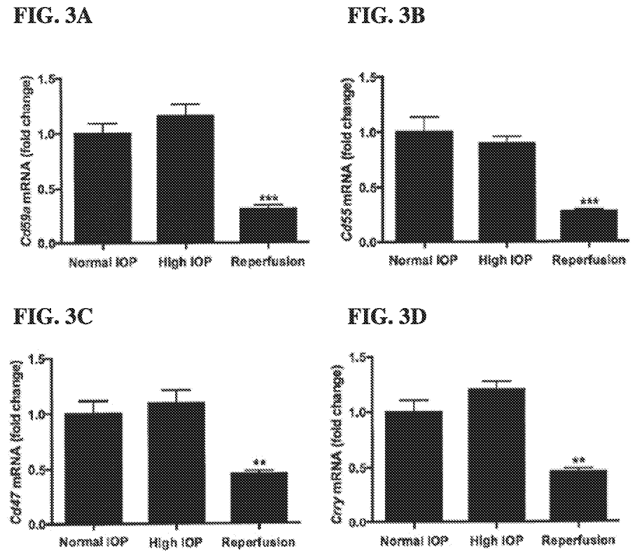

IR injury causes a decrease in complement inhibitor expression in the mouse retina. Cd55 is down regulated in HUVECs under hypoxic conditions, and it is down regulated in neovessels lacking bloodflow. To further investigate these findings, along with other complement regulators, we adopted a mouse model of retinal I/R. I/R injury is characterized by a loss of blood flow to the retina, thus a loss of shear stress and oxygen in the vessels and tissue. qRT-PCR analysis showed a significant decrease in Cd47, Cd55, Cd59a, and Crry (FIGS. 3A-D.) expression in the mouse retina after 6 hours of reperfusion following ischemia.

### Complement Mediated Neuronal Cell Death in I/R Injury

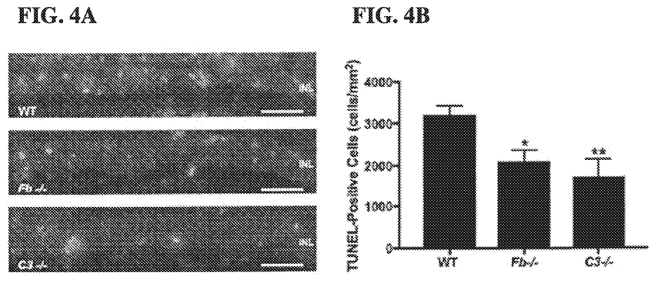

To investigate whether complement mediates retinal cell death in I/R injury, mice deficient in complement proteins C3 and Fb were used to block alternative complement pathway functioning. Fb knock-out is protective against neuronal cell death in I/R, as shown in FIGS. 4A and 4B. TUNEL staining showed that Fb knockout was protective against complement mediated neuronal cell death in the INL of the retina (FIGS. 4A and 4B). C3 knock-out is also protective against neuronal cell death in I/R, as shown in FIGS. 4A and 4B. TUNEL Staining also showed that C3 knockout is protective against neuronal cell death in the INL of the retina (FIGS. 4A and 4B). The data show that the decrease in retinal production of complement regulators during I/R injury causes complement-mediated neuronal cell death in the retina. Thus, complement is a therapeutic target to reduce or prevent neuronal cell death.

### Protective Effect of Shear Stress Against Complement

Shear stress increases the expression of complement inhibitors, and has a protective effect against complement mediated cell death in HRECs. In detail, increased sheer stress led to a significantly higher expression of Cd47, Cd55, and Cd59 compared to the static control. This increase in expression also had an anti apoptotic effect on HRECs when incubated in mouse serum, used as a source of foreign complement, in a model of shear stress. For example, Cd59 is up regulated in HUVEC cells that are exposed to shear-stress. The lack of shear stress during I/R injury causes a decrease in complement inhibitor expression.

### Inhibition of the Alternative Complement Pathway Protects Against and Reduces Neuronal Cell Death

The complement system is part of the innate immune response in the body and is in a constant state of low-level expression. This expression and activation is regulated by a variety of proteins, which prevent over activation of complement. An imbalance in activation leads to destruction of foreign or self cells. The studies described herein were carried out to elucidate the role of complement in I/R injury, and determine its utility as therapeutic target to reduce retinal damage and neuronal cell death.

In I/R injury there is a partial or total reduction of blood flow to the tissue. This reduction in blood flow leads to a reduction in complement regulatory protein expression. The data showed that in a mouse model of I/R there is a reduction in complement regulatory proteins after 6 h of reperfusion.

The data demonstrate that the compositions and methods described herein reduce or inhibit complement-mediated death of neuronal cells during disease or injury.

### Example 2: The Alternative Complement System Mediates Cell Death in Retinal Ischemia Reperfusion Injury

Ischemia reperfusion (IR) injury induces retinal cell death and contributes to visual impairment. The complement cascade plays a key role in IR injury in several systemic diseases. However prior to the invention, the role of the complement pathway in the ischemic retina has not been investigated. Studies were carried out to determine if the alternative complement cascade plays a role in retinal IR injury, and identify which components of the pathway mediate retinal degeneration in response to IR injury. To accomplish this, we utilized the mouse model of retinal IR injury, wherein the intraocular pressure (IOP) is elevated for 45 minutes, collapsing the retinal blood vessels and inducing retinal ischemia, followed by IOP normalization and subsequent reperfusion. We found that mRNA expression of complement inhibitors complement receptor 1-related gene/protein-y (Crry), Cd55 and Cd59a was down-regulated after IR. Moreover, genetic deletion of complement component 3 (C3^{-/-}) and complement factor b (Fb^{-/-}) decreased IR-induced retinal apoptosis. Because vascular dysfunction is central to IR injury, we also assessed the role of complement in a model of shear stress. In human retinal endothelial cells (HRECs), shear stress up-regulated complement inhibitors Cd46, Cd55 and Cd59, and suppressed complement-mediated cell death, indicating that a lack of vascular flow, commonly observed in IR injury, allows for complement mediated attack of the retinal vasculature. These results indicated that in retinal IR injury, the alternative complement system is activated by suppression of complement inhibitors, leading to vascular dysfunction and neuronal cell death.

### Ischemia Reperfusion Injury and Ocular Immune Activity

Ischemia reperfusion (IR) injury is a complex pathophysiological phenomenon that is initiated by the loss of blood flow to a tissue and resultant ischemia followed by the subsequent return of blood flow, which results in oxidative stress and downstream cytotoxic inflammatory effects. Retinal ischemia is a major cause of damage in ocular diseases including glaucoma, central retinal vein occlusion and diabetic retinopathy. Current therapeutic approaches for IR injury focus on returning retinal blood flow through the use of anticoagulants, vasodilators, and laser treatment, or on alleviating oxidative stress using free radical scavengers. Currently, no clinically available therapeutic modalities are focused on reperfusion-associated inflammatory damage, a major contributor to IR injury. In order to develop these important approaches to treatment, it is essential to identify which inflammatory mediators contribute to retinal IR-induced inflammatory damage. The data described herein identifies components of the alternate complement pathway that contribute to retinal IR-induced inflammatory damage and provides therapeutic methods to reduce such damage and confer a clinical benefit to subjects afflicted with retinal ischemic damage associated with injury, chronic condition, and/or glaucoma.

Ocular immune activity, including activation of the complement cascade, is dramatically increased in multiple forms of ocular IR injury. The complement system is comprised of a complex network of proteins that work together to induce an inflammatory response that signals for removal of nonfunctional self-cells and non-self-cells. There are three complement pathways (classical, lectin and alternative) that activate the central component of the complement system, C3, which subsequently activates a final common pathway facilitating cellular clearance. Evolutionarily, the alternative complement pathway is the oldest of the complement activation pathways, so is broadly conserved. The alternative complement pathway is a key mediator of endogenous immune surveillance and tissue homeostasis. To monitor tissue homeostasis, the alternative complement pathway remains in a constant state of low-level activation, which allows for continuous probing of cells. In contrast, the classical and lectin pathways do not maintain this low-level activation.

Activation of the alternative complement pathway occurs through the spontaneous hydrolysis of an internal thioester bond within C3. Further continuation of the pathway is allowed only by complement factor b (Fb), which binds to C3b deposited in the membrane of a target cell. These components form the C3 convertase enzyme, promoting the cleavage of C3 into C3a and C3b, creating a positive feedback activation loop that is down-regulated by complement inhibitors such as Crry/CD46, Cd55 and Cd59.

Soluble and cell-bound regulators of complement help to protect healthy host tissue from self-recognition and serve to prevent activation of a complement response. However, damaged or diseased host cells may down-regulate membrane bound inhibitors of complement, allowing targeted clearance. An imbalance between complement recognition and initiation on healthy host cells can lead to unregulated complement activation and subsequent cellular damage. Thus, activation of complement not only helps defend the host against pathogens, but also has the potential to affect self-tissues in a both beneficial (protective autoimmunity) and detrimental manner (autoimmunity).

The alternative complement pathway is an important regulator of ocular health in proliferative retinopathy and retinal detachment. Within the ocular microenvironment, the alternative complement pathway maintains low levels of constitutive activity to ensure the intermittent probing of host cells. In normal conditions, host cells normally express endogenous membrane inhibitors of complement (e.g., Cd55) to protect against targeting by the complement system, these inhibitors are down-regulated in response to host cell injury allowing for their targeted removal by the complement system.

Studies were carried out to investigate the role of the alternative complement system in IR injury-induced neuronal damage and vascular dysfunction associated with IR injury. The role of the complement cascade in IR injury has been investigated in many tissues including the kidney, intestine, lung and heart. However prior to the invention, the role of the complement cascade in IR injury has not been established. The studies and data described herein indicate complement pathway inhibition is useful as a therapy to alleviate IR-induced cell death and injury.

Ischemia is categorized by a lack of blood flow to tissue associated with vascular dysfunction. Most studies of ischemic disease have focused upon the ramifications of decreased oxygen and nutrient availability. However, blood flow also causes shear stress, which is the frictional force exerted on vessel wall endothelial cells. Physiologic levels of shear stress are important for normal endothelial cell function. Shear regulates the Kruppel Like Factor 2 (KLF-2) transcription factor, which regulates molecules important to endothelial cell homeostasis including vascular endothelial growth factor (VEGF), endothelial Nitric Oxide Synthase (eNOS), and CD59. Thus, given the role of vascular dysfunction associated with IR injury and complement's role in targeting injured self-cells we were interested in investigating the role decreased shear stress had on complement inhibitor expression in retinal endothelial cells. The down-regulation of complement inhibitors, occurring as a result of decreased shear stress in IR injury, could contribute to vascular damage and dysfunction through overactivation of the complement system.

### Circulation was Obstructed in the IR Model

For the retinal IR model, we applied a widely-used method, which causes retinal ischemia by increasing the intraocular pressure (IOP) above the ocular perfusion pressure. In this model, the anterior chamber, located directly behind the cornea, is cannulated with a needle connected to a sterile saline reservoir to increase intraocular pressure and collapse retinal blood vessels, inducing retinal ischemia. The blood supply deficiency results in oxygen and nutrient deprivation of the retina, causing retinal cell death.

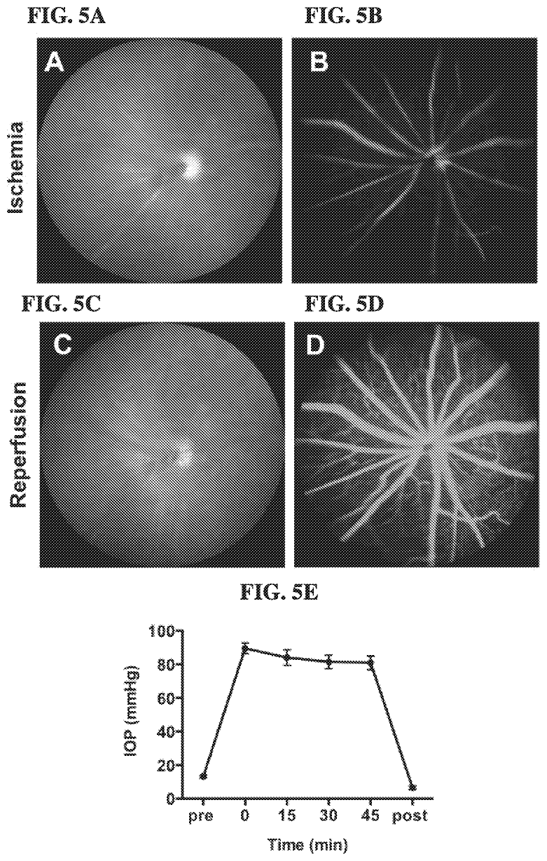

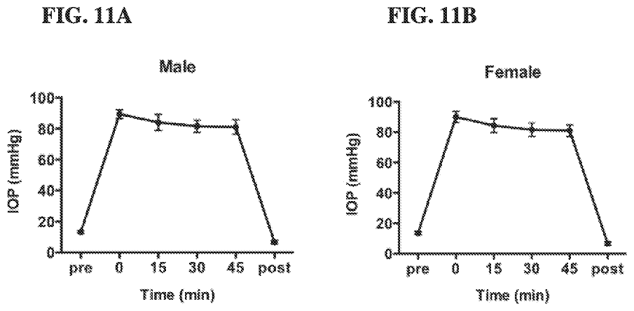

To investigate the obstruction of the blood flow by IR surgery, we recorded color fundus videos and fluorescein angiography (FA) videos during the IR operation. Immediately after the elevation of the IOP, constriction of vessels and whitening of the whole retina were observed in the color fundus video (FIG. 5A). In FA, remarkable slowing of blood flow and a lack of choroidal filling were observed (FIG. 5B). When the needle was removed from the anterior chamber, blood flowed in from central retinal artery instantaneously, allowing retinal vessels to revert to normal width and the retina to regain its original color (FIG. 5C). FA demonstrated that blood flow reverted to a normal velocity, and that choroidal filling was restored (FIG. 5D). We next assessed IOP during the IR operation. IOP was measured before the IR surgery, 0 min, 15 min, 30 min and 45 min after the onset of IR, and immediately after the removal of the needle. Mean IOP before the surgery (13±0.3 mmHg) was within normal range. By the onset of the IR, mean IOP increased to 90±1 mmHg and remained over 80 mmHg during the surgery (84±1 mmHg at 15 min, 82±1 mmHg at 30 min and 81±1 mmHg at 45 min) (FIG. 5E). IOP decreased immediately upon removal of the needle from the anterior chamber. There was no significant difference between male and female animals at all time points (FIG. 11A and FIG. 11B). Taken together, these results indicate that the IR model impaired blood supply from both choroidal and retinal circulation, and that ischemia was maintained during the 45-minute surgery.

Inner Nuclear Layer Apoptosis was Maximal at 24 h after Reperfusion

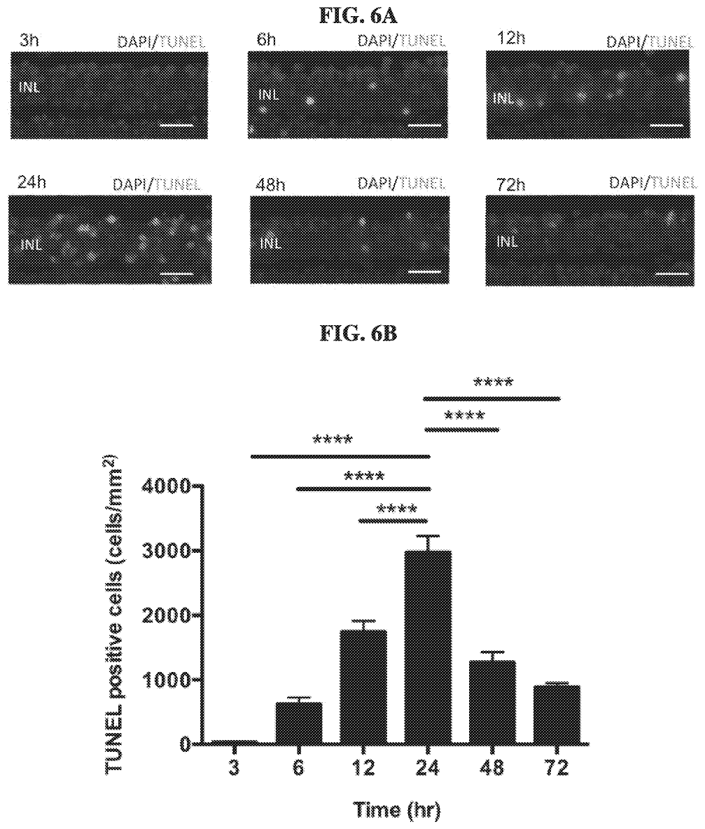

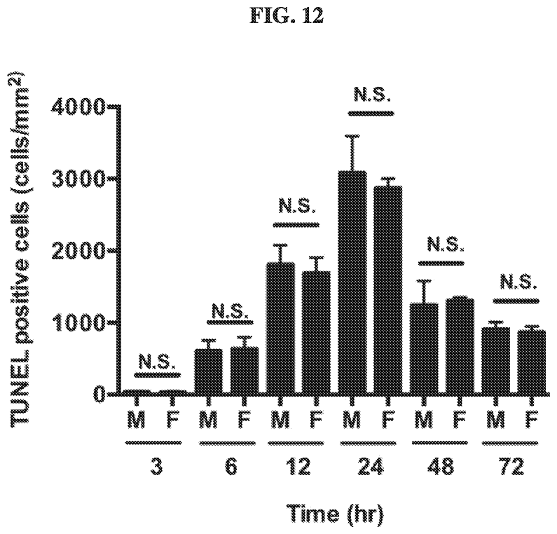

In the mouse model of IR injury, the inner nuclear layer (INL) of the retina is more prone to reproducible irreversible cell loss caused by IR injury, thus we sought to assess cell death in the INL for our studies. INL status is assessed using standard opththalmologic methods, such as Optical coherence tomography (OCT), a non-invasive imaging test that uses light waves to take cross-section pictures of the retina each of the retina's distinctive layers to map, evaluate, and measure their thickness. These measurements are used to diagnose and identify subjects for treatment for retinal ischemic injury, glaucoma and other diseases of the retina using the methods and compositions described herein. In addition to OCT, a visual acuity test is also used and a second method to evaluate INL status and is a decrease in visual acuity is reflective of pathologic cell death in the INL. To assess changes in INL cell death following acute ocular hypertension, we performed terminal deoxynucleotidyl transferase dUTP nick end labeling (TUNEL) staining of retinas at 3 h, 6 h, 12 h, 24 h, 48 h and 72 h after reperfusion. The number of apoptotic cells peaked 24 h after reperfusion (2976±252 cells/mm²), significantly higher than other time points (FIG. 6A and FIG. 6B). There was no difference between male and female mice at all the time points (FIG. 12). Based on this result, we analyzed cell death 24 h after reperfusion in subsequent mechanistic studies.

### Retinal Expression of Complement Inhibitors is Down-Regulated in Response to IR

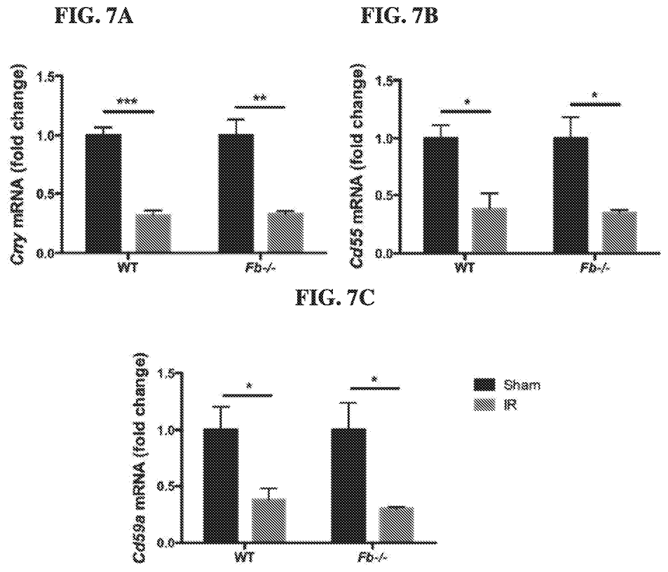

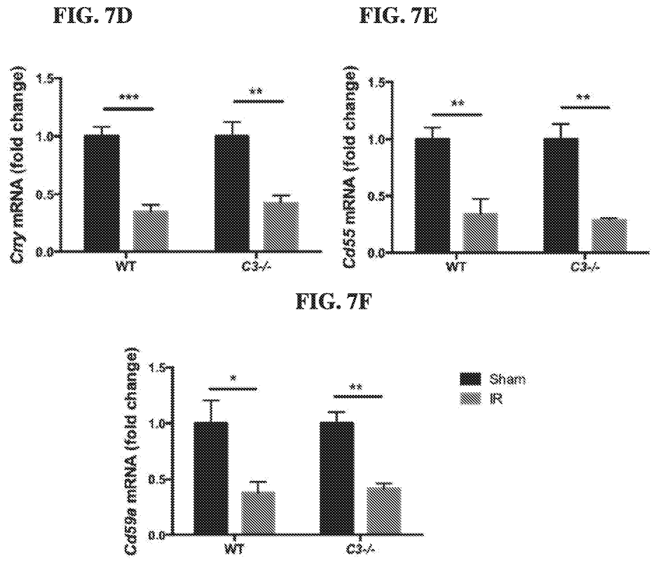

The complement system acts as an immune surveillance system to discriminate among healthy host tissue, cellular debris, apoptotic cells, varying its response in reaction to cellular injury. Generally, normal healthy host cells endogenously express membrane-bound complement inhibitors to prevent self-recognition and targeting by the complement system, thereby protecting themselves from complement-mediated cell death. However, damaged host cells down-regulate complement inhibitors, allowing for targeted clearance of damaged cells. To investigate whether the complement pathway is involved in IR injury-induced retina cell death, we evaluated the expression of complement inhibitors complement receptor 1-related gene/protein-y (Crry), Cd55 and Cd59a in C57BL6 mice, Fb^{-/-} mice and C3^{-/-} mice at 6 hours after IR surgery or sham surgery. The complement inhibitors were significantly decreased in the retina after IR surgery in all three strains (FIGS. 7A-7F). This result indicates that the complement cascade may be activated by suppression of complement inhibitors in IR injury.

### Retinal Cell Death was Suppressed in Complement Pathway-Deficient Mice

The complement system is comprised of three pathways (classical, lectin, and alternative pathways). All three pathways activate the central complement component 3 (C3). Subsequent entry into a final terminal pathway results in membrane attack complex (MAC) formation, which creates a pore in the cell membrane and leads to cell lysis and death.

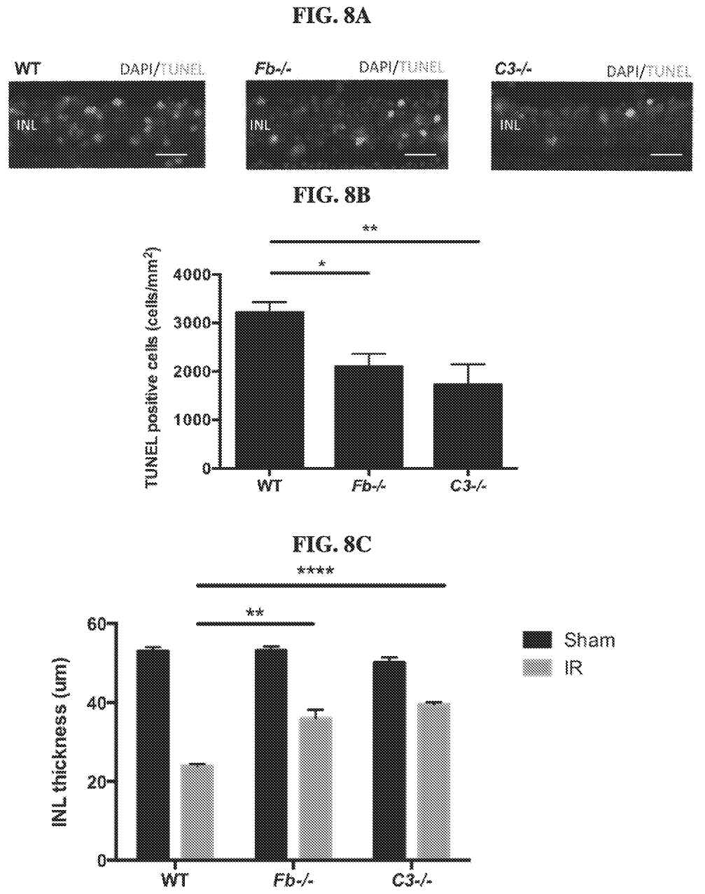

To elucidate the role of the complement system in retinal cell death caused by IR, we performed retinal TUNEL staining in IR-injured C3^{-/-} mice relative to wild-type controls. C3^{-/-} mice lack the central C3 protein required for all three complement pathways. Cell death was quantified at 24 hours after IR, the peak of cell death. The number of TUNEL-positive cells in C3^{-/-} mice (1727±418 cells/mm²) decreased by 46% compared to C57BL6 mice (3213±216 cells/mm²) (FIG. 8A and FIG. 8B).

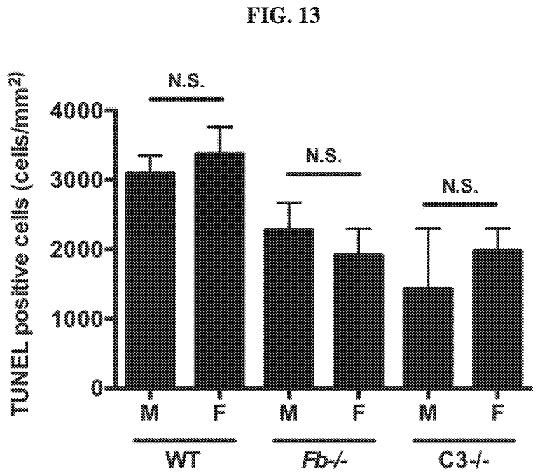

To further elucidate the role of the complement system in retinal IR injury, we next measured retinal cell death in complement factor b-deficient (Fb^{-/-}) mice, which are deficient in the effector of the alternative pathway. The number of apoptotic cells in Fb^{-/-} mice (2098±268 cells/mm²) was down-regulated by 35% compared to C57BL6 mice (FIG. 8A and FIG. 8B). There was no difference between male and female mice of both strains (FIG. 13).

In order to evaluate the long-term effect of IR injury, we analyzed INL thickness in C57BL6 mice, Fb^{-/-} mice and C3^{-/-} mice 7 days after IR surgery or sham surgery. When compared to C57BL6 mice (24±1 μm), a reduction of INL thickness after IR injury was suppressed by 47% in Fb−/− (38±2 μm) and by 63% in C3−/− mice (40±1 μm). This demonstrated that IR injury induces morphologic changes in the retina, and revealed the long-term protective effect of lack of C3 or Fb (FIG. 8C). These results indicate that the alternative complement pathway contributes to the retinal cell death caused by IR injury.

### Shear Stress has a Protective Effect Against Complement Mediated Cell Death

The main factor in IR injury pathology is the loss of blood flow, leading to vascular dysfunction as well as neuronal damage. Most investigations of ischemic disease have focused on the consequences of resultant oxygen and nutrient deprivation. However, loss of blood flow also results in loss of shear stress in endothelial cells lining vessel walls, which is important in endothelial cell biology. Shear stress may also play a role in IR injury. Endothelial cells are known producers of complement, and we investigated whether shear stress affected complement activation in endothelial cells.

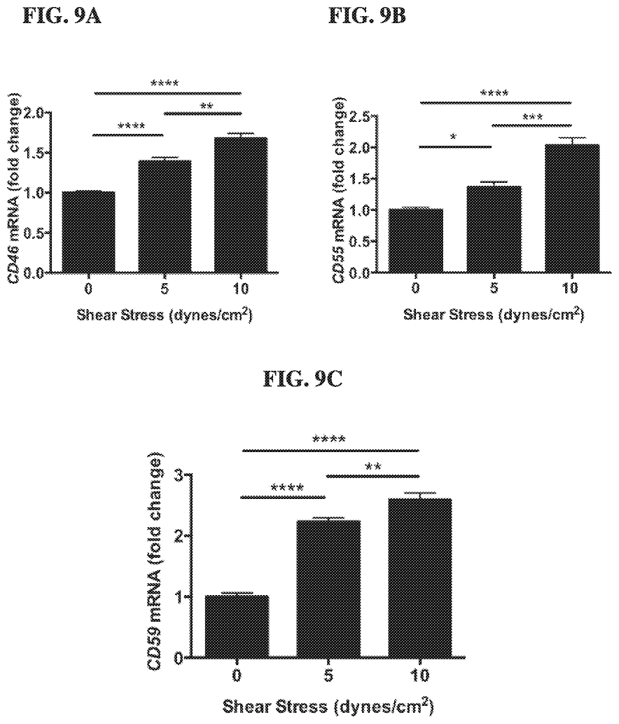

To investigate the effect of shear stress on complement inhibitors, we evaluated the expression of Cd46, Cd55 and Cd59 in human retinal endothelial cells (HRECs). The expression of complement inhibitors was determined by real-time polymerase chain reaction (RT-PCR) after exposure to orbital shear stress (0, 5, or 10 dynes/cm²) for 24 hours. All three complement inhibitors were significantly up-regulated by shear stress in proportion to the relative amount of shear stress applied (FIG. 9A-FIG. 9C).

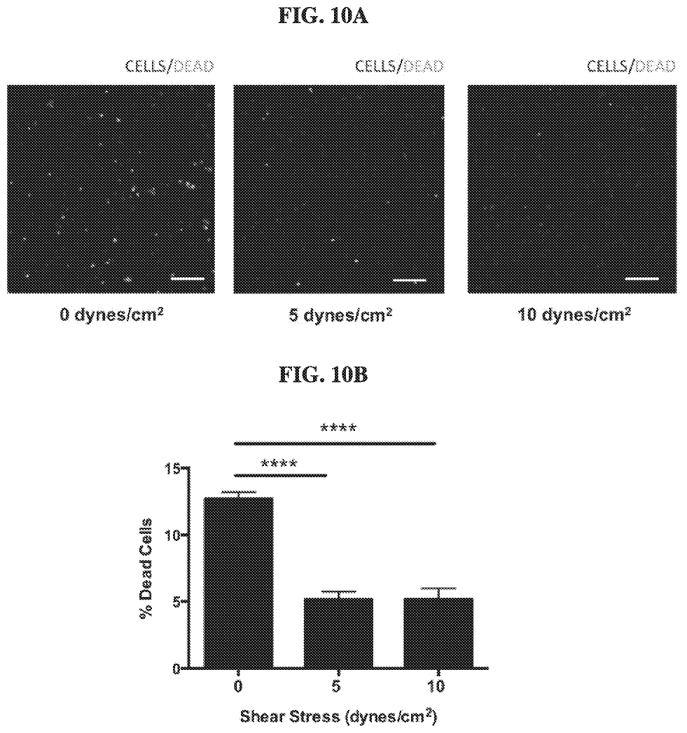

We next assessed whether shear stress may protect against complement-mediated cell death. HRECs were exposed to shear stress (0, 5, or 10 dynes/cm²) for 24 hours during incubation in media containing 1% mouse serum as a source of foreign complement. Cells were stained with a viability imaging kit, and the percentage of dead cells was calculated. The percentage of HRECs undergoing cell death was significantly suppressed by 60% in both 5 and 10 dynes/cm² magnitudes (5±1% and 5±1%, respectively) compared to 0 dynes/cm² magnitude (13±1%) (FIG. 10A and FIG. 10B). These data indicate that shear stress contributes to protection from complement-mediated cell death by up-regulating expression of complement inhibitors.

### Alternative Complement-Mediated Cell Death in IR Injury

IR injury is a well-established model of retinal vascular occlusion diseases such as acute glaucoma and diabetic retinopathy. Although the elevation of IOP is well known to affect retinal perfusion, the IOP during the ischemic phase of IR surgery ranges from 60 to 120 mmHg, and the reservoir height used is not standardized, meaning that there may be significant variation between investigators. Furthermore, ischemia and reperfusion are usually examined indirectly by observation of the iris, episcleral veins or the reflex from the fundus. Few studies have focused on observing the ischemia and reperfusion directly, meaning that the degree of ischemia and reperfusion attained could vary between groups. In this study, we demonstrated that blood supply from both the choroidal and retinal circulation is obstructed immediately by elevating the IOP to 80 mmHg, and reperfused by the removal of the needle.

While several studies have reported the time course of apoptosis in the IR model, the conclusion is still controversial. In our IR model, TUNEL-positive cells in INL were found as early as 3 hours after the reperfusion, peaked at 24 hours, and decreased gradually over subsequent time points. This result is consistent with a previous report evaluating apoptotic cells in the INL of IR-injured rat retinas. The earliest evaluated time point in most prior studies was 6 or 12 hours post-reperfusion. We also measured retinal apoptosis 3 hours post-reperfusion, and found that apoptosis began to occur earlier than previously reported.

The complement system is a mediator of systemic IR injury. However, little is known about complement-mediated cell death in retinal IR injury. In the present study, we demonstrated that compliment inhibitors Crry, Cd55 and Cd59a were suppressed early on in the retina (6 hours) after IR injury in C57BL6 mice, Fb^{-/-} mice and C3^{-/-} mice. These inhibitors are regarded as critical negative regulators of the complement system due to their ability to modulate all three complement pathways. Crry inhibits C3 convertase, CD55 inhibits both C3 convertase and C5 convertase, and CD59 inhibits formation of the MAC. Our findings indicate that down-regulation of complement inhibitor expression increases activation of the complement pathway, leading to formation of the MAC complex and eventual death of retinal cells.

In our retinal IR model, cell death in INL was suppressed in C3^{-/-} mice compared to wild-type (WT) mice. Furthermore, a similar reduction in the amount of retinal cell death was observed in Fb^{-/-} mice. Consistent with these results, when the thickness of the INL was measured 7 days following IR injury in WT, C3^{-/-} and Fb^{-/-} mice, complement deficient strains were found to have significant protection against INL degeneration compared to WT mice in response to IR injury. As factor B is exclusive to the alternative pathway, these results indicate that the complement system, especially the alternative pathway, likely contributes to IR injury-induced retinal cell death. We previously found that the classical and lectin pathways play a minor role in oxygen-induced retinopathy model and photoreceptor cell death retinal detachment model, using classical pathway knockout mice (C1q^{-/-}) and lectin pathway knockout mice (Mbl A/C^{-/-}). Thus, it would be interesting to assess the contribution of these pathways in this IR model in a future study.

Our current in vitro experiments showed that apoptotic cell death was suppressed by shear stress in HRECs, demonstrating that shear stress protects human umbilical vein endothelial cells (HUVECs) from apoptosis. Furthermore, we also demonstrated that complement inhibitors Cd46, Cd55 and Cd59 are up-regulated by shear stress.

In conclusion, we found that complement system plays a key role in IR injury-induced retinal cell death. Our data indicate that the lack of blood flow may contribute to the suppression of complement inhibitors, leading to apoptotic cell death via activation of the complement system.

The following materials and methods were used to generate the data described in Example 2.

### Animals

C57BL6 mice (stock no. 000664) at 8 wk of age were obtained from The Jackson Laboratory (Bar Harbor, Me., USA). Fb^{-/-} and C3^{-/-} mice were also used in the studies. All animals used were between the ages of 8 and 9 weeks for all studies performed.

### Mouse Model of Retinal Ischemia Reperfusion (IR) and Glaucoma

Mice were anesthetized with Avertin (T4, 840-2; 2,2,2 tribromoethanol; Sigma-Aldrich, St. Louis, Mo., USA, in isoamyl alcohol, A730-1; ThermoScientific, Waltham, Mass., USA), and deep anesthesia was examined by a toe pinch test. Pupils were dilated with 1% tropicamide, and topical anesthesia (1 drop of proparacaine hydrochloride) was applied to cornea. The anterior chamber of the left eye was cannulated with a 30-gauge needle connected to a line infusing sterile saline. The IOP was raised by elevating the saline reservoir to cause ischemia. After 45 minutes of ischemia, the needle was withdrawn to allow reperfusion. IOP was measured 0, 15, 30 and 45 minutes after induction of ischemia, before and after the surgery using a TonoLab tonometer (icare, Vantaa, Finland). For sham surgery, a 30-gauge needle was inserted into the anterior chamber without elevating the IOP. Mice were kept on a heating pad until fully awake, as examined by upright mobility. Mice were euthanized at various time points after IR, and their retinas were prepared as described below.

### Fundus Video and Fluorescein Angiography

Fundus color video and FA during the IR surgery were taken using the Micron III Retinal Imaging Microscope (Phoenix Research Laboratories, Pleasanton, Calif., USA). For FA, 0.1 ml of 2% fluorescein sodium (Akorn, Lake Forest, Ill., USA) was injected intraperitoneally. Images were taken by attaching the cornea covered with 2.5% Goniovisc (HUB Pharmaceuticals, Rancho Cucamonga, Calif., USA) to the Micron camera lens using StreamPix software (Phoenix Research Laboratories, Pleasanton, Calif., USA).

### TUNEL Cell Death Assay

Immediately after enucleation, mouse eyes were placed in optimum cutting temperature (OCT) compound (Tissue Tek, 4583, Torrance, Calif., USA), and quickly frozen by submerging in a beaker of isopropanol chilled by dry ice. All eyes were cut into 10 μm sections with four sections per eye.

TUNEL was performed using the ApopTag Fluorescein In Situ Apoptosis Kit (S7110; Millipore, Billerica, Mass., USA) following the manufacturers instructions. The sections were coverslipped with 4′,6-diamidino-2-phenylindole (DAPI) containing medium (H-1200; Vector Laboratories, CA, USA). All images were obtained using an AxioVision microscope (Zeiss, Chester, Va., USA), and the TUNEL cell counter plugin in Fiji image analysis software was used to automatically calculate the area and number of TUNEL-positive cells. When using the TUNEL Cell Counter plugin, the retina area was selected manually and the threshold sensitivity was changed to high in all the images, while all other settings remained unchanged. Eight images were taken in the midperiphery of each retina using a 20× objective lens.

### Hematoxylin and Eosin (H & E) Staining

Mice were euthanized on day 7 following IR or sham surgery, and eyes were enucleated. All eyes were fixed in 4% paraformaldehyde and paraffin embedded. Sections (6-μm thick) were cut parallel to the maximal circumference of the eye ball through the optic nerve and stained with hematoxylin and eosin (H&E). Inner nuclear layer (INL) thickness was measured in eight areas within 200-500 μm from the optic nerve, and the mean value was calculated.

### Cell Culture

Human retinal endothelial cells (HRECs) were purchased from Cell Sytems, Inc. (ACBRI 181; Kirkland, Wash., USA) and grown in EGM-2 Growth Medium with SingleQuots (Bulletkit CC-3162; Lonza, Basel, Switzerland) supplemented with 1% L-glutamine and penicillin-streptomycin. They were grown to 90% confluence in T75s coated with 0.2% gelatin under the following incubator conditions: 5% CO2, 37° C., and 95% humidity. The cells had been cultured for 7-9 passages.

### Shear Stress Model

Cells were seeded into three wells of a 6-well plate at a density of 500,000 cells/well. For each experiment, one plate was used for each magnitude of shear stress. Cells were synchronized by culturing in starvation medium (EBM-2 basal medium (CC-3156; Lonza), 5% calf serum, 25 mM N-2-hydroxyethylpiperazine-N′-2-ethanesulfonic acid (HEPES), L-glutamine, and penicillin-streptomycin) for 24 h. After 24 h, fresh starvation medium was added and plates were exposed to orbital shear stress for 24 h using orbital shakers (Orbi-Shaker JR BT300; Benchmark Scientific, Sayreville, N.J., USA) set to either ˜5 (150 rpm) or ˜10 (240 rpm) dynes/cm² inside an incubator. Cells of the same passage were placed in the same incubator as the static control. Shear stress was approximated using the formula Tₘₐₓ=α\sqrt{ρη(2πf)³}, where α is the radius of orbital rotation (0.95 cm), ρ is the density of the medium (1.0 g/ml), η is the viscosity of the medium poise (7.5×10⁻³ dynes·sec/cm²), and f is the frequency of rotation (rotations/second).

### Live/Dead Assay

HRECs were cultured in starvation medium supplemented with 1% mouse serum, while being exposed to shear stress (0, 5, or 10 dynes/cm²) for 24 h. The percentage of dead cells was determined using the ReadyProbes Cell Viability Imaging Kit (ThermoFisher Scientific, Waltham, Mass., USA). Three representative images of the peripheral area were taken per well using Zeiss Zen imaging software. Without any post processing, Fiji image analysis software was used to automatically count the number of dead cells (green) and total cells (blue) using the particle analyzer tool. The number of dead cells and total cells of the three images taken for each well were averaged to calculate the percent cell death per well.

RNA Isolation and cDNA Preparation

For retinal gene expression analysis, eyes were collected 6 hours after IR surgery or sham surgery, and dissected retinas were flash frozen in liquid nitrogen. For HRECs, cells were treated with trypsin, pelleted by centrifugation, and flash frozen. RNA was collected using the RNeasy Mini Kit (Qiagen, Hilden, Germany) following the manufacturers instructions. Total RNA was measured using the NanoDrop 2000 Spectrophotometer (ThermoFisher Scientific). cDNA was normalized prior to reverse transcription with SuperScript III and random hexamer primers (Invitrogen, Carlsbad, Calif., USA).

### Real-Time PCR Analysis

RT-PCR was performed using 1 μl of cDNA and a StepOnePlus Real-Time PCR System (Applied Biosystems, Waltham, Mass., USA). Primers to Crry (Mm00787529_s1, ThermoFischer Scientific), Cd55 (Mm00438377_m1, ThermoFischer Scientific), Cd59a (Mm00483149_m1, ThermoFischer Scientific) were used for mouse retinas, and primers to Cd46 (Hs00611257_m1, ThermoFischer Scientific), Cd55 (Hs00892618_m1, ThermoFischer Scientific) and Cd59 (Hs00174141_m1; ThermoFischer Scientific) were used for HRECs with TaqMan Universal PCR Master Mix (4304437, ThermoFischer Scientific) and probes were used in combination with TaqMan Universal PCR Master Mix (4304437; ThermoFisher Scientific). All data was normalized to β-actin.

### Statistical Analysis

Data were analyzed using one-way ANOVA. Results are expressed as mean±SEM. Significance was set at P<0.05.

### Description of Figures in Example 2

FIGS. 5A-E show that obstruction of blood flow and the intraocular pressure (IOP) changes during IR surgery. Representative images of fundus color video (FIG. 5A) and fluorescein angiography (FIG. 5B) during the ischemia phase of IR surgery. Representative images of fundus color video (FIG. 5C) and fluorescein angiography (FIG. 5D) during the reperfusion phase of IR surgery. (FIG. 5E) IOP was measured before the IR surgery, 0, 15, 30 and 45 minutes after the initiation of the ischemia phase and immediately after the reperfusion. n=12 eyes/time point. The TonoLab was used under general anesthesia. IOP before surgery was normal (12.4 mmHg). By comparison, onset of the IR, IOP was increased to 90 mmHg and remained over 80 mmHg. Data are presented as the mean±SEM.

FIGS. 6A-B show that IR induced apoptosis peaked 24 hours after reperfusion. (FIG. 6A) Representative images of TUNEL labeling of retinal cross-sections at 3, 6, 12, 24, 48 and 72 hours after IR injury co-labeled with DAPI (blue) and TUNEL (green). Scale bars=20 μm. INL, inner nuclear layer. (FIG. 6B) Quantification of TUNEL-positive cells in the INL at 3, 6, 12, 24, 48 and 72 hours after IR injury. n=8-10 eyes/time point. Images were taken at two areas of the retina per section for a total of eight images per eye. The area of the INL and number of TUNEL-positive cells were automatically calculated by TUNEL cell counter software. Data are presented as the mean±SEM. ****P<0.0001 vs. 24 hour. IR mouse model: 80-90 mmHg for 45 min.

FIGS. 7A-C show that complement inhibitors gene expression was down-regulated in IR retina. The mRNA levels of complement inhibitors, Crry (FIG. 7A, FIG. 7D), Cd55 (FIG. 7B, FIG. 7E) and Cd59a (FIG. 7C, FIG. 7F) in the retina of wild type control mice (WT), complement factor b-deficient mice (Fb−/−) and complement component 3-deficient mice (C3−/−) at 6 hours after IR injury were quantified by RT-PCR, and were normalized to sham surgery. n=4 eyes/group. Data are presented as the mean±SEM. *P<0.05 vs. sham, **P<0.01 vs. sham, ***P<0.001 vs. sham.

FIGS. 8A-B show that IR induced retinal cell death and degeneration is suppressed in complement-deficient mice. (FIG. 8A) Representative images of TUNEL labeling of retinal cross-sections at 24 hours after IR injury in wild type control mice (WT), complement factor b-deficient mice (Fb−/−) and complement component 3-deficient mice (C3−/−), colabeled with DAPI (blue) and TUNEL (green). Scale bars=20 μm. INL, inner nuclear layer. (FIG. 8B) Quantification of TUNEL-positive cells in the INL of WT control, Fb−/− and C3−/− mice at 24 hours after IR injury. The number of TUNEL-positive cells in Fb−/− mice decreased by 34.7% compared to C57BL6 mice. The number of TUNEL-positive cells in C3−/− mice decreased by 46.3% compared to C57BL6 mice. n=11-14 eyes/group. Data are presented as the mean±SEM. *P<0.05 vs. WT, **P<0.01 vs. WT. (FIG. 8C) The thickness of the INL was measured 7 days after IR injury or sham surgery. n=6 eyes/group. Data are presented as the mean±SEM. **P<0.01 vs. WT, ****P<0.0001 vs. WT.

FIGS. 9A-C show that complement inhibitors were up-regulated in HRECs under shear stress. (FIG. 9A)-(FIG. 9C) The mRNA levels of complement inhibitors, CD46 (FIG. 9A), Cd55 (FIG. 9B) and Cd59 (FIG. 9C) in HRECs were quantified by RT-PCR after exposure to orbital shear stress (5 or 10 dynes/cm2) for 24 hours, and were normalized to control (0 dynes/cm2). The expression of complement inhibitors was increased in proportion to the magnitude of the shear stress applied. Data are the average of 4 independent experiments. Data are presented as mean±SEM. *P<0.05, **P<0.01, ***P<0.001, ****P<0.0001.

FIGS. 10A-C show that complement mediated cell death was suppressed in HRECs by shear stress. (FIG. 10A) Representative images of HRECs stained with a cell viability imaging kit after exposure to orbital shear stress (0, 5 or 10 dynes/cm2) for 24 hours during incubation in media with foreign complement. Cells (blue), dead cells (green). Scale bars=1200 μm. (FIG. 10B) The percentage of dead cells in HRECs after exposure to orbital shear stress (0, 5 or 10 dynes/cm2) for 24 hours with media containing foreign complement. The cell death was significantly suppressed in HRECs exposed to shear stress. Data are the average of 5 independent experiments. Data are presented as the mean±SEM. ****P<0.0001 vs. 0 dynes/cm2. IR mouse model: 80-90 mmHg for 45 min. n=4-5 eyes/time point.

FIGS. 11A-11B show that no gender difference was observed in intraocular pressure (IOP) changes during ischemia reperfusion (IR) surgery. Mean IOP before, after and during the 45-minute IR surgery in male (FIG. 11A) and female (FIG. 11B) mice. n=6 eyes/time point. Data are presented as the mean±SEM.

FIG. 12 shows that no gender difference was observed in time course of ischemia reperfusion (IR) induced apoptosis. Quantification of TUNEL-positive cells in the INL at the indicated time points after IR. TUNEL indicates terminal deoxynucleotidyl dUTP nick end labeling; M, male; and F, female. n=4-5 eyes/time point. Data are presented as the mean±SEM. N.S., not significant.

FIG. 13 shows that no gender difference was observed in ischemia reperfusion (IR) induced apoptosis in complement pathway-deficient mice. Quantification of TUNEL-positive cells in the INL in WT control, Fb−/− and C3−/− mice after IR. TUNEL, terminal deoxynucleotidyl dUTP nick end labeling; INL, inner nuclear layer; and WT, wild type control. n=5-8 eyes/group. Data are presented as the mean±SEM. N.S., not significant.

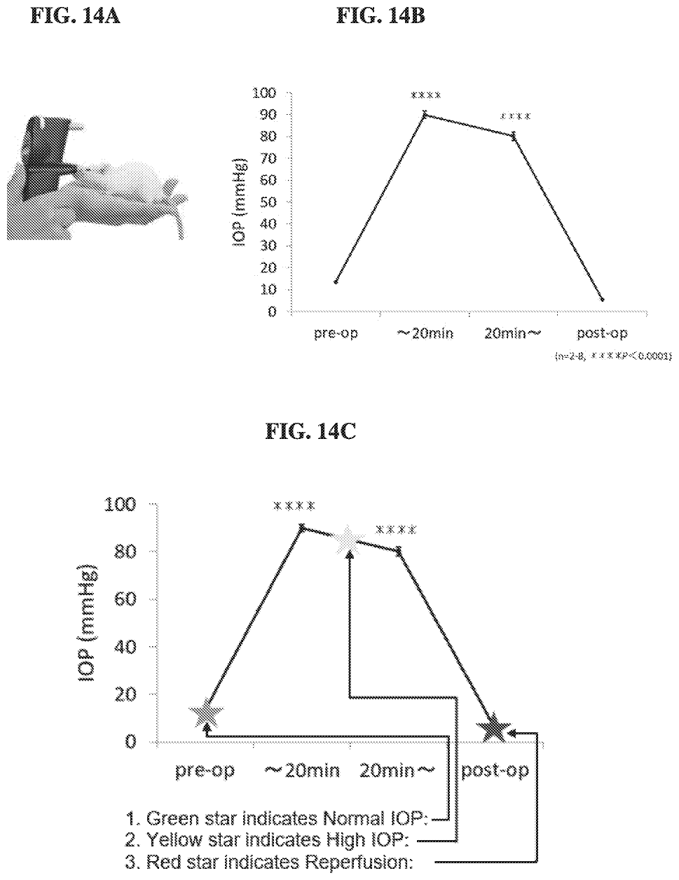

FIGS. 14A-FIG. 14C. The IOP measurement (FIG. 14A) and the TOP trend (FIG. 14B and FIG. 14C) during the I/R injury model. Upon removal of the needle from the anterior segment the IOP is decreases immediately, allowing reperfusion to occur. FIG. 14C shows the experimental procedure based on the IOP trend. Three experimental groups are designed: (i) the normal IOP group for which the needle is inserted for 45 min but the saline reservoir is not elevated; (ii) the High IOP group for which the needle is inserted for 45 minutes and the saline bag is elevated; and (iii) the reperfusion group for which the needle is inserted for 45 minutes and the saline bag is elevated followed by 6 hours of reperfusion before sample collection.

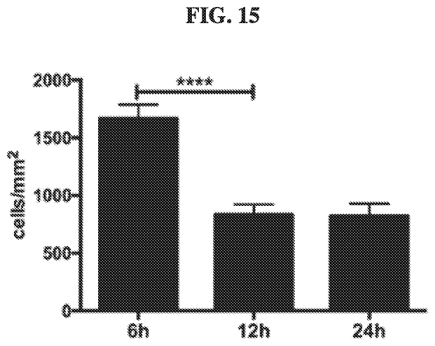

FIG. 15 shows a cell death time course in I/R.

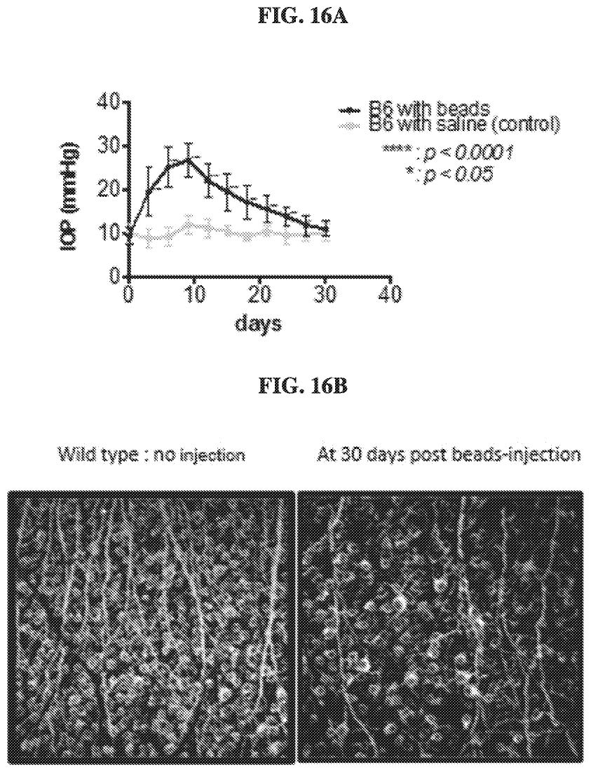

FIG. 16A and FIG. 16B shows IOP and RGCs in the conventional I/R model of the wild-type (C57BL/6J) mice. In the conventional model, IOP gradually increased and reached a maximum at 10 days post injection (FIG. 16A), with a value of about 25 mmHg. The images below (FIG. 16B) are results of Tuj-1 staining, showing decreased Tuj-1 staining in wild-type mice at 1 month post injection. There was minimal retinal ganglion cell damage using this original method.

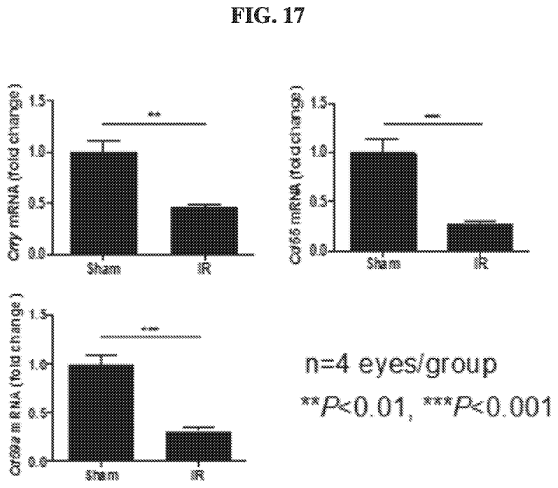

FIG. 17 shows membrane bound inhibitors of complement are suppressed in retinal I/R injury. Down regulation of membrane bound inhibitors of complement facilitate complement targeting of stressed or dying cells in IR.

As set forth above, Applicants showed that complement regulator protein expression is regulated by shear stress in human retinal endothelial cells, and complement regulator protein expression in the mouse retina is decreased after I/R injury. Furthermore, knock-out of complement proteins C3 and Fb is protective against I/R injury in the mouse retina. The alternative complement system facilitates the removal of diseased cells in the ocular microenvironment. Together, Applicants demonstrate that complement system plays a key role in IR injury-induced retinal cell death and provides a therapeutic target against vascular and retinal insult.

### Ocular Hypertension Models of Glaucoma

Clinical Glaucoma

Glaucoma is one of the leading causes of blindness worldwide. The chief complaint of patients with glaucoma is a concentric contraction of the visual field. There are many different forms of glaucoma that differ significantly in clinical presentation and disease progression. However, they all share a common endpoint, which is the loss of retinal ganglion cells.

Glaucomatous Optic Neuropathy: Pathogenesis

Glaucoma is a multifactorial disorder. A variety of factors which can contribute to retinal ganglion cell death were identified from the clinical and basic science settings. In glaucoma, apoptosis of RGCs is mainly induced by the elevation of IOP. However, the essential cause of glaucoma still remains unclear.

Glaucoma Models: Comparison

Established models were used for investigating retinal ganglion cell death. These models are classified into two categories, genetic and artificial. See, for example, at the table below:

| Classi- | | | | |
|---|---|---|---|---|
| fication | Genetic models | | Artificial models | |
| Type | DBA2NNia | DBA2J | PC | Bead |
| | | | (photo- | occlusion |
| | | | coagulation | |
| | | | of iris) | |
| | | | induced | |
| Mechanism | Angle | Pigmentary | Angle | Angle |
| | closure | glaucoma | closure | closure |
| Genetic | Unknown | Gpnmb, | n/a | n/a |
| abnormalities | | Tyrp 1 | | |
| IOP | <20 mmHg | Maximum | around | 20-30 mmHg |
| | | <20 mmHg | 30 mmHg | |
| Persistence | Elevation of | Gradually | Maximum: | Maximum: |
| of IOP | IOP in | elevated | 2 Mo after | around 10 |
| | P8 Mo- | | surgery | days after |
| | P12 Mo | | | surgery |
| Optic disc | After 15 mo | Around 22 | After 3 mo | 1 mo |
| abnormality | | mo | | |
| Other | | Iris atrophy | | |
| | | iris | | |
| | | dislocation, | | |
| | | cataract, NV | | |

Many researchers have been using the DBA2J model. However, it takes a long time to induce severe degeneration in retinal ganglion cells. Among the artificial models, the PC induced model is sometimes used; however, it takes a couple of months to induce strong damage in retinal ganglion cells and the success rate is inconsistent. Compared to these models, the microbead occlusion model has a relatively short induction time. Applicants used the microbead occlusion model.

Microbead Occlusion Model

In this system, 3 microliters of beads mixed in a saline suspension are injected into the anterior chamber. Eventually the beads migrate to the angle, and prevent the drainage of aqueous humor, resulting in elevated IOP and subsequent damage of RGCs. However, the original method has a problem of the limited elevation of IOP, which causes minimal extent of damage to RGCs. Leakage usually occurs at the incision site immediately after injection, making it hard to re-produce the same injection across experiments. In addition, Anti-β-III-tubulin (Tuj-1)Ab (antibody) stains both axons and cell bodies and thus it is hard to estimate the amount of damage in RGCs around the disc because the axonal stain around the disc is very strong, making it difficult to identify individual cells.

Accordingly, Applicants employed the modified OH methods. For example, a modified OH method using hyaluronic acid includes the steps of (i) injecting beads suspended in hyaluronic acid at the angle incision, such that elevated IOP will help self-seal wound, using a 31G Glass needle; and (ii) using glue at incision site to adjust the inconsistent injecting volume. The model was assessed by the brain-specific homeobox/POU domain protein 3A (Brn3a) Quant, i.e., Brn3a stain of the whole retina. The area is divided into midperiphery and periphery and then 4 areas are selected in each region and the damage estimated by automatic cell count system using Image-J. Ocular hypertension models of glaucoma are also analyzed by, e.g., induction of the model, IOP measurement, immunohistochemistry (IHC) and quantification.

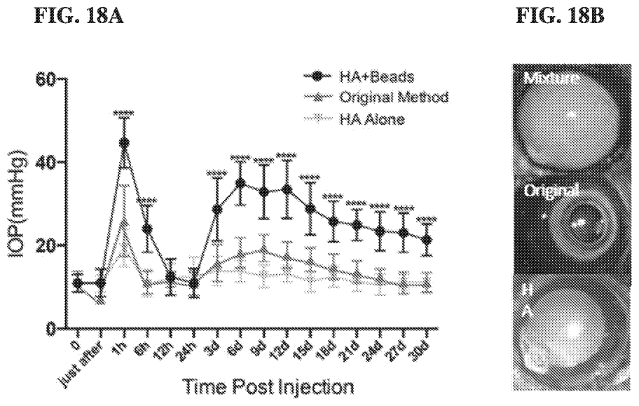

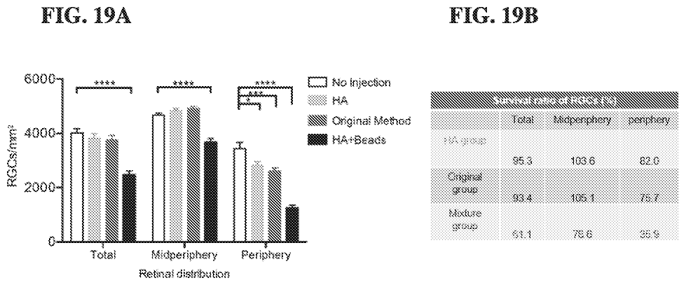

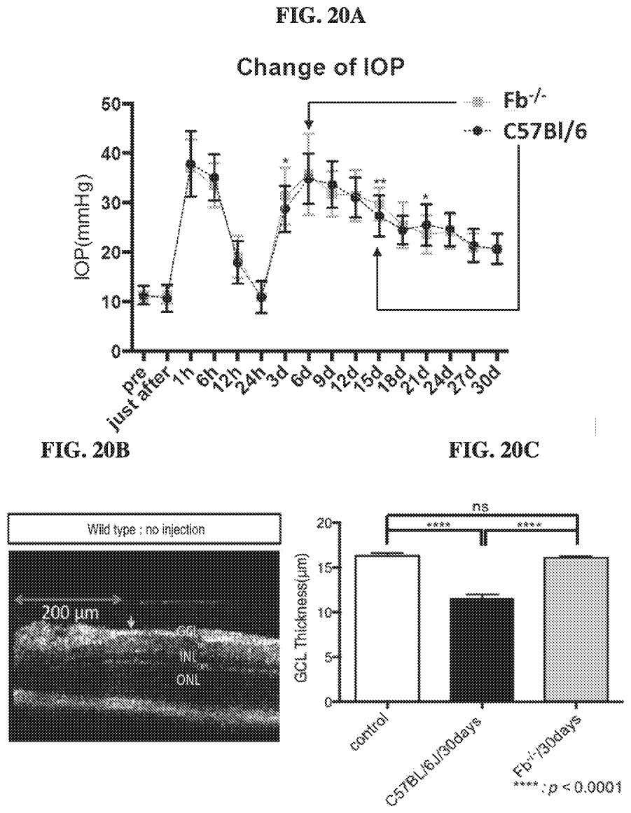

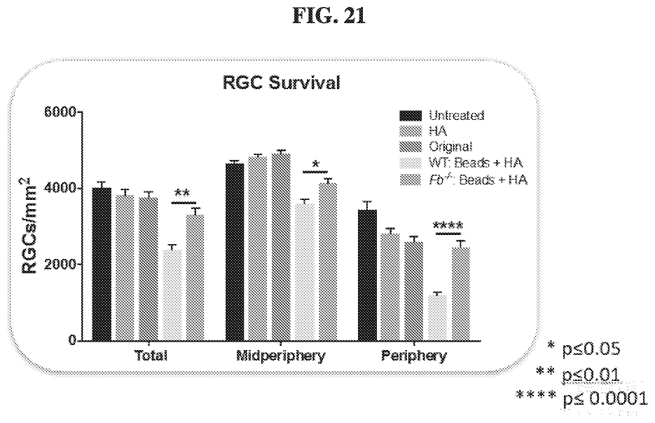

FIG. 18A and FIG. 18B shows IOP in the modified ocular hypertension (OH) model of glaucoma, supporting the advantages of the modified OH method over the original method. The advantages of the modified OH method is further demonstrated by the disease severity observed in the models (FIG. 19A and FIG. 19B). Using the modified OH method, the RGC loss in alternative complement deficient mice following OH compared with the wild-type mice is shown in FIGS. 20A, 20B, 20C and 21.

### Optic Nerve Crush (ONC) as a Model for Clinical Optic Nerve Injury

ONC is carried out by (i) approaching from superior; (ii) exposing optic nerve; (iii) crushing optic nerve for 10 sec using forceps; and (iv) developing retrograde RGC degeneration.

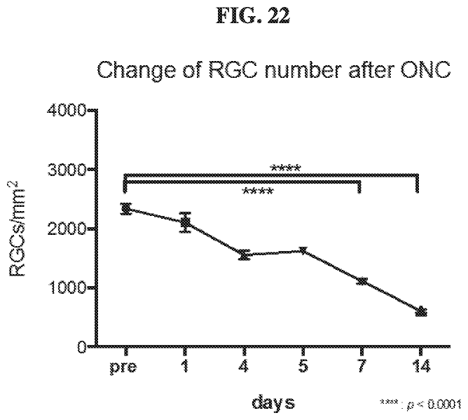

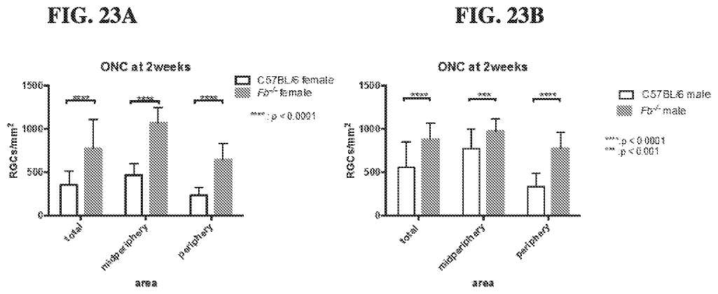

FIG. 22 shows the RGC loss following optic nerve crush (ONC). The RGC loss in female (FIG. 23A) and male (FIG. 23B) alternative complement deficient mice following ONC, compared with the wild-type mice are shown in FIGS. 23A and 23B.

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “subject,” “patient,” “individual,” and the like as used herein are not intended to be limiting and can be generally interchanged. That is, an individual described as a “patient” does not necessarily have a given disease, but may be merely seeking medical advice.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, “treating” encompasses, e.g., inhibition, regression, or stasis of the progression of a disorder. Treating also encompasses the prevention or amelioration of any symptom or symptoms of the disorder. As used herein, “inhibition” of disease progression or a disease complication in a subject means preventing or reducing the disease progression and/or disease complication in the subject.

As used herein, a “symptom” associated with a disorder includes any clinical or laboratory manifestation associated with the disorder, and is not limited to what the subject can feel or observe.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. Such sequences that are at least about 80% identical are said to be “substantially identical.” In some embodiments, two sequences are 100% identical. In certain embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids or nucleotides in length. In certain embodiments, the identity exists over a region that is at least about 50 amino acids in length, or more preferably over a region that is 100 to 500, 100 to 200, 150 to 200, 175 to 200, 175 to 225, 175 to 250, 200 to 225, 200 to 250 or more amino acids in length.

For sequence comparison, typically one sequence acts as a reference sequence, to which test sequences are compared. In various embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In various embodiments, a comparison window is the entire length of one or both of two aligned sequences. In some embodiments, two sequences being compared comprise different lengths, and the comparison window is the entire length of the longer or the shorter of the two sequences. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

In various embodiments, an algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information, as known in the art. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

1. A method for reducing or inhibiting retinal cell death in a subject comprising retinal ischemic injury, the method comprising administering to an ischemic retinal tissue of said subject a composition comprising an agent that inhibits or reduces alternative complement pathway activity.

2. The method of claim 1, wherein said retinal cell death comprises neuronal cell death.

3. The method of claim 1, wherein said subject is identified as comprising retinal ischemia or retinal ischemia reperfusion injury.

4. The method of claim 1, wherein said agent inhibits or reduces the activity of C3, Factor B (Fb), Factor C5 or Factor D, in said neuronal tissue.

5. The method of claim 1, wherein said agent inhibits or reduces the activity of at least one selected from C3, Factor B (Fb), properdin (Factor p), factor Ba, factor Bb, factor D, C2, C2a, C3a, C3b, C5, C5a, C5b, C6, C7, C8, C9, and C5b-9 in said retinal tissue.

6. The method of claim 1, wherein said agent inhibits or reduces the activity, transcription stability, translation, modification, localization, cleavage, or function of a polynucleotide or polypeptide encoding any one of the selected from C3, factor B (Fb), properdin (Factor p), factor Ba, factor Bb, factor D, C2, C2a, C3a, C3b, C5, C5a, C5b, C6, C7, C8, C9, and C5b-9 in said neuronal tissue.

7. The method of claim 1, wherein said agent comprises an antibody or an antigen-binding fragment thereof, a small molecule, a polynucleotide, or a polypeptide.

8. The method of claim 1, wherein said agent comprises a serine protease inhibitor, a soluble form of a complement receptor, a humanized monoclonal anti-complement antibody or antibody fragment, a complement component inhibitor, a nucleic acid expression vector encoding anti-complement polypeptides, or an anaphylatoxin receptor antagonist.

9. The method of claim 1, wherein said composition is administered to said subject by intraocular injection, intravitreal administration, topical administration, a subconjunctival administration, intranasal administration, intrathecal administration, systemic administration, or directly into the brain.

10. The method of claim 1, wherein said neuronal cell death is associated with retinal Ischemia reperfusion (IR) injury or glaucoma.

11. The method of claim 1, wherein said cell death comprises apoptosis.

12. The method of claim 1, wherein said cell death comprises caspase-mediated apoptosis.

13. The method of claim 1, wherein said cell is a retinal ganglion cell.

14. A pharmaceutical composition for reducing or inhibiting retinal cell death in a subject comprising retinal ischemic injury, said composition comprising an agent that inhibits or reduces alternative complement pathway activity.

15. The composition of claim 16, wherein said agent inhibits or reduces the activity of C3, Fb, Factor D, or C5 in said neuronal tissue.

16. The composition of claim 16, wherein said agent inhibits or reduces the activity of at least one selected from C3, Fb, properdin (Factor p), factor Ba, factor Bb, factor D, C2, C2a, C3a, C3b, C5, C5a, C5b, C6, C7, C8, C9, and C5b-9 in said neuronal tissue.

17. The composition of claim 16, wherein said agent inhibits or reduces the transcription stability, translation, modification, localization, cleavage, or function of a polynucleotide or polypeptide encoding any one of the selected from C3, Fb, properdin (Factor p), factor Ba, factor Bb, factor D, C2, C2a, C3a, C3b, C5, C5a, C5b, C6, C7, C8, C9, and C5b-9 in said neuronal tissue.

18. The composition of claim 14, wherein said agent comprises an antibody or an antigen-binding fragment thereof, a small molecule, a polynucleotide, or a polypeptide.

19. The composition of claim 14, wherein said agent comprises a serine protease inhibitor, a soluble form of a complement receptor, a humanized monoclonal anti-complement antibody or antibody fragment, a complement component inhibitor, a nucleic acid expression vector encoding anti-complement polypeptides, or an anaphylatoxin receptor antagonist.

20-28. (canceled)

29. The method of claim 1, wherein said administering step is carried out during prior to an ischemic ocular event, at the time of reperfusion of ischemic retinal tissue, within 3 hours of perfusion, within 12 hours of perfusion.
